SEQUENCE LISTING (31 sequences)

SEQ ID NO: 1 - DNA, 1383 bp, Artificial Sequence
  Synthetic Primer
atgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacgcggaatacacgaacatcttcctcaaatttggatctggctatgtaagtggctggggaagagtcttccacaaaggggcatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcact

SEQ ID NO: 2 - protein, 461 aa, Artificial Sequence
  Synthetic Peptide
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADAEYTNIFLKFGSGYVSGWGRVFHKGASALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 3 - DNA, 1383 bp, Artificial Sequence
  Synthetic Primer
atgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcgcatttggatctggctatgtaagtggctggggaagagtcttccacaaaggggcatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcact

SEQ ID NO: 4 - protein, 461 aa, Artificial Sequence
  Synthetic Peptide
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLAFGSGYVSGWGRVFHKGASALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 5 - DNA, 1383 bp, Artificial Sequence
  Synthetic Primer
atgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacgcggaatacacgaacatcttcctcgcatttggatctggctatgtaagtggctggggaagagtcttccacaaaggggcatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcact

SEQ ID NO: 6 - protein, 461 aa, Artificial Sequence
  Synthetic Primer
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADAEYTNIFLAFGSGYVSGWGRVFHKGASALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 7 - DNA, 1383 bp, Homo sapiens
atgcagcgcgtgaacatgatcatggcagaatcaccaggcctcatcaccatctgccttttaggatatctactcagtgctgaatgtacagtttttcttgatcatgaaaacgccaacaaaattctgaatcggccaaagaggtataattcaggtaaattggaagagtttgttcaagggaaccttgagagagaatgtatggaagaaaagtgtagttttgaagaagcacgagaagtttttgaaaacactgaaagaacaactgaattttggaagcagtatgttgatggagatcagtgtgagtccaatccatgtttaaatggcggcagttgcaaggatgacattaattcctatgaatgttggtgtccctttggatttgaaggaaagaactgtgaattagatgtaacatgtaacattaagaatggcagatgcgagcagttttgtaaaaatagtgctgataacaaggtggtttgctcctgtactgagggatatcgacttgcagaaaaccagaagtcctgtgaaccagcagtgccatttccatgtggaagagtttctgtttcacaaacttctaagctcacccgtgctgagactgtttttcctgatgtggactatgtaaattctactgaagctgaaaccattttggataacatcactcaaagcacccaatcatttaatgacttcactcgggttgttggtggagaagatgccaaaccaggtcaattcccttggcaggttgttttgaatggtaaagttgatgcattctgtggaggctctatcgttaatgaaaaatggattgtaactgctgcccactgtgttgaaactggtgttaaaattacagttgtcgcaggtgaacataatattgaggagacagaacatacagagcaaaagcgaaatgtgattcgaattattcctcaccacaactacaatgcagctattaataagtacaaccatgacattgcccttctggaactggacgaacccttagtgctaaacagctacgttacacctatttgcattgctgacaaggaatacacgaacatcttcctcaaatttggatctggctatgtaagtggctggggaagagtcttccacaaagggagatcagctttagttcttcagtaccttagagttccacttgttgaccgagccacatgtcttcgatctacaaagttcaccatctataacaacatgttctgtgctggcttccatgaaggaggtagagattcatgtcaaggagatagtgggggaccccatgttactgaagtggaagggaccagtttcttaactggaattattagctggggtgaagagtgtgcaatgaaaggcaaatatggaatatataccaaggtatcccggtatgtcaactggattaaggaaaaaacaaagctcact

SEQ ID NO: 8 - protein, 461 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 9 - protein, 235 aa, Homo sapiens
VVGGEDAKPGQFPWQVVLNGKVDAFCGGSIVNEKWIVTAAHCVETGVKITVVAGEHNIEETEHTEQKRNVIRIIPHHNFNAAINTYNHDIALLELDEPLVLNSYVTPICIADKEYTNIFLKFGSGYVSGWGRVFHKGRSALVLQYLRVPLVDRATCLRSTKFTITNNMFCAGFHEGGRDSCQGDSGGPHVTEVEGTSFLTGIISWGEECAMKGKYGIYTKVSRYVNWIKEKTKLT

SEQ ID NO: 10 - protein, 235 aa, Bos taurus
VVGGEDAERGQFPWQVLLHGEIAAFCGGSIVNEKWVVTAAHCIKPGVKITVVAGEHNTEKPEPTEQKRNVIRAIPYHSYNASINKYSHDIALLELDEPLELNSYVTPICIADRDYTNIFLKFGYGYVSGWGKVFNRGRSASILQYLKVPLVDRATCLRSTKFSIYSHMFCAGYHEGGKDSCQGDSGGPHVTEVEGTSFLTGIISWGEECAMKGKYGIYTKVSRYVNWIKEKTKLT

SEQ ID NO: 11 - protein, 227 aa, Sus scrofa
IVGGENAKPGQFPWQVLLNGKIDAFCGGSIINEKWVVTAAHCIEPGVKITVVAGEYNTEETEPTEQRRNVIRAIPHHSYNATVNKYSHDIALLELDEPLTLNSYVTPICIADKEYTNIFLKFGSGYVSGWGRVFNRGRSATILQYLKVPLVDRATCLRSTKVTIYSNMFCAGFHEGGKDSCLGDSGGPHVTEVEGTSFLTGIISWGEECAVKGKYGIYTKVSRYVNW

SEQ ID NO: 12 - protein, 234 aa, Canis familiaris
VVGGKDAKPGQFPWQVLLNGKVDAFCGGSIINEKWVVTAAHCIEPDVKITVAGEHNTEKREHTEQKRNVIRTILHHSYNATINKYNHDIALLELDEPLTLNSYVTPICIADREYSNIFLKFGSGYVSGWGRVFNKGRSASILQYLKVPLVDRATCLRSTKFTIYNNMFCAGFHEGGKDSCQGDSGGPHVTEVEGISFLTGIISWGEECAMKGKYGIYTKVSRYVNWIKEKTKLT

SEQ ID NO: 13 - protein, 235 aa, Oryctolagus cuniculus
IYGGENAKPGQFPWQVLLNGKVEAFCGGSIINEKWVVTAAHCIKPDDNITVVAGEYNIQETENTEQKRNVIRIIPYHKYNATINKYNHDIALLELDKPLTLNSYVTPICIANREYTNIFLNFGSGYVSGWGRVFNRGRQASILQYLRVPFVDRATCLRSTKFTIYNNMFCAGFDVGGKDSCEGDSGGPHVTEVEGTSFLTGIISWGEECAIKGKYGVYTRVSWYVNWIKEKTKLT

SEQ ID NO: 14 - protein, 224 aa, Ovis aries
VVGGEDAARGQFPWQVLLHGEIAAFCGGSIVNEKWVVTAAHCIKPGVKITYVAGEHNTEKPEPTEQKRNVIRAIPYHGYNASINKYSHDIALLELDEPLELNSYVTPICIADREYTNIFLKFGYGYVSGWGRVFNRGRSASILQYLKVPLVDRATCLRSTKFTIYNHMFCAGYHEGGKDSCQGDSGGPHVTEVEGTSFLTGIISWGEECAMKGKYGIYTKVSRY

SEQ ID NO: 15 - protein, 227 aa, Cavia porcellus
VVGGEDAKPGQFPIQVLLNGETEAFCGGSIVNEKWIVTAAHCILPGIKIEVVAGKHNIEKKEDTEQRRNVTQIILHHSYNASFNKYSHDIALLELDKPLSLNSYVTPICIANREYTNIFLKFGAGYVSGWGKLFSQGRTASILQYLRVPLVDRATCLRSTKFTIYNNMFCAGFHEGGRDSCQGDSGGPHVTEVEGTNFLTGIISWGEECAMKGKYGIYTKVSRYVHW

SEQ ID NO: 16 - protein, 235 aa, Mus musculus
VVGGENAKPGQIPWQVILNGEIEAFCGGAIINEKWIVTAAHCLKPGDKIEVVAGEYNLDKKEDTEQRRNVIRTIPHHQYNATINKYSHDIALLELDKPLILNSYVTPICVANREYTNIFLKFGSGYVSGWGKVFNKGRQASILQYLRVPLVDRATCLRSTTFTIYNNMFCAGYREGGKDSCEGDSGGPHVTEVEGTSFLTGIISWGEECAMKGKYGIYTKVSRYVNWIKEKTKLT

SEQ ID NO: 17 - protein, 227 aa, Rattus rattus
VVGGENAKPGQIPWQVILNGEIEAFCGGAIINEKWIVTAAHCLKPGDKLEVVAGEHNIDEKEDTEQRRNVIRTIPHHQYNATINKYSHDIALLELDKPLILNSYVTPICVANKEYTNIFLKFGSGYVSGWGKVFNKGRQASILQYLRVPLVDRATCLRSTKFSIYNNMFCAGYREGGKDSCEGDSGGPHVTEVEGTSFLTGIISWGEECAMKGKYGIYTKVSRYVNW

SEQ ID NO: 18 - protein, 230 aa, Bos taurus
IVNGEEAVPGSWPWQVSLQDKTGFHFCGGSLINENWVVTAAHCGVTTSDVVVAGEFDQGSSSEKIQKLKIAKVFKNSKYNSLTINNDITLLKLSTAASFSQTVSAVCLPSASDDFAAGTTCVTTGWGLTRYTNANTPDRLQQASLPLLSNTNCKKYWGTKIKDAMICAGASGVSSCMGDSGGPLVCKKNGAWTLVGIVSWGSSTCSTSTPGVYARVTALVNWVQQTLAAN

SEQ ID NO: 19 - protein, 224 aa, Rattus rattus
IIGGVESIPHSRPYMAHLDIVTEKGLRVICGGFLISRQFVLTAAHCKGREITVILGAHDVRKRESTQQKIKVEKQIIHESYNSVPNLHDIMLLKLEKKVELTPAVNVVPLPSPSDFIHPGAMCWAAGWGKTGVRDPTSYTLREVELRIMDEKACVDYRYYEYKFQVCVGSPTTLRAAFMGDSGGPLLCAGVAHGIVSYGHPDAKPPAIFTRVSTYVPTINAVIN

SEQ ID NO: 20 - protein, 240 aa, Sus scrofa
VVGGTEAQRNSWPSQISLQYRSGSSWAHTCGGTLIRQNWVMTAAHCVDRELTFRVVVGEHNLNQNNGTEQYVGVQKIVVHPYWNTDDVAAGYDIALLRLAQSVTLNSYVQLGVLPRAGTILANNSPCYITGWGLTRTNGQLAQTLQQAYLPTVDYAICSSSSYWGSTVKNSMVCAGGNRGVSGCQGDSGGPLHCLVNGQYAVHGVTSFVSRLGCNVTRKPTVFTRVSAYISWINNVIASN

SEQ ID NO: 21 - protein, 218 aa, Homo sapiens [sequence omitted]

SEQ ID NO: 22 - protein, 234 aa, Rattus rattus
IVGGYKCEKNSQPWQVAVINEYLCGGVLIDPSWVITAAHCYSNNYQVLLGRNNLFKDEPFAQRRLVRQSFRHPDYIPLIVTNDTEQPVHDHSNDLMLLHLSEPADITGGVKVIDLPTKEPKVGSTCLASGWGSTNPSEMVVSHDLQCVNIHLLSNEKCIETYKDNVTDVMLCAGEMEGGKDTCAGDSGGPLICDGVLQGITSGGATPCAKPKTPAIYAKLIKFTSWIKKVMKEN

SEQ ID NO: 23 - protein, 234 aa, Sus scrofa
IIGGRECEKNSHPWQVAIYHYSSFQCGGVLVNPKWVLTAAHCKNDNYEVWLGRHNLFENENTAQFFGVTADFPHPGFNLSADGKDYSHDLMLLRLQSPAKITDAVKVLELPTQEPELGSTCEASGWGSIEPGPDDFEFPDEIQCVQLTLLQNTFCAHBHBDKVTESMLCAGYLPGGKDTCMGDSGGPLICNGMWQGITSWGHTPCGSANKPSIYTKLIFYLDIINBTITENDGK

SEQ ID NO: 24 - protein, 223 aa, Bos taurus
IVGGYTCGANTVPYQVSLNSGYHFCGGSLINSQWVVSAAHCYKSGIQVRLGEDNINVVEGNEQFISASKSIVHPSYNSNTLNNDIMLIKLKSAASLNSRVASISLPTSCASAGTQCLISGWGNTKSSGTSYPDVLKCLKAPILSDSSCKSAYPGQITSNMFCAGYLEGGKDSCQGDSGGPVVCSGKLQGIVSWGSGCAQKNKPGVYTKVCNYVSWIKQTIASN

SEQ ID NO: 25 - protein, 259 aa, Homo sapiens
IVEGSDAEIGMSPWQVMLFRKSPQELLCGASLISDRWVLTAAHCLLYPPWDKNFTENDLLVRIGKHSRTRYERNIEKISMLEKIYIHPRYNWRENLDRDIALMKLKKPVAFSDYIHPVCLPDRETAASLLQAGYKGRVTGWGNLKETWTANVGKGQPSVLQVVNLPIVERPVCKDSTRIRITDNMFCAGYKPDEGKRGDACEGDSGGPFVMKSPFNNRWYQMGIVSWGEGCDRDGKYGFYTHVFRLKKWIQKVIDQFGE

SEQ ID NO: 26 - protein, 254 aa, Homo sapiens [sequence omitted]

SEQ ID NO: 27 - protein, 253 aa, Homo sapiens
IVGGQECKDGECPWQALLINEENEGFCGGTILSEFYILTAAHCLYQAKRFKVRVGDRNTEQEEGGEAVHEVEVVIKHNRFTKETYDFDIAVLRLKTPITFRMNVAPACLPERDWAESTLMTQKTGIVSGFGRTHEKGRQSTRLKMLEVPYVDRNSCKLSSSFIITQNMFCAGYDTKQEDACQGDSGGPHVTRFKDTYFVTGIVSWGEGCARKGKYGIYTKVTAFLKWIDRSMKTRGLPKAKSHAPEVITSSPL

SEQ ID NO: 28 - protein, 238 aa, Homo sapiens
IVGGTASVRGEWPWQVTLHTTSPTQRHLCGGSIIGNQWILTAAHCFYGVESPKILRVYSGILNQAEIAEDTSFFGVQEIIIHDQYKMAESGYDIALLKLETTVNYADSQRPISLPSKGDRNVIYTDCWVTGWGYRKLRDKIQNTLQKAKIPLVTNEECQKRYRGHKITHKMICAGYREGGKDACKGDSGGPLSCKHNEVWHLVGITSWGEGCAQRERPGVYTNVVEYVDWILEKTQAV

SEQ ID NO: 29 - protein, 242 aa, Homo sapiens
VVGGLVGLRGAHPYIAALYWGHSFCAGSLIAPCWVLTAAHCLQDRPAPEDLTVVLGQERRNHSCEPCQTLAVRSYRLHEAFSPVSYQHDLALLRLQEDADGSCALLSPYYQPVCLPSGAARPSETTLCQVAGWHQFEGAEEYASFLQEAQVPFLSLERCSAPDVHGSSILPGMLCAGFLEGGTDACQGDSGGPLVCEDQAAERRLTLQGIISWGSGCGDRNKPGVYTDVAYYLAWIREHTVS

SEQ ID NO: 30 - protein, 250 aa, Homo sapiens
LIDGKMTRRGDSPWQVVLLDSKKKLACGAVLIHPSWVLTAAHCMDESKKLLVRLGEYDLRRWEKWELDLDIKEVFVHPNYSKSTTDNDIALLHLAQPATLSQTIVPICLPDSGLAERELNQAGQETLVTGWGYHSSREKEAKRNRTFVLNFIKIPVVPHNECSEVMSNMVSENMLCAGILGDRQDACEGDSGGPMVASFHGTWFLVGLVSWGEGCGLLHNYGVYTKVSRYLDWIHGHIRDKEAPQKSWAP

SEQ ID NO: 31 - protein, 230 aa, Homo sapiens
VVGGCVAHPHSWPWQVSLRTRFGMHFCGGTLISPEWVLTAAHCLEKSPRPSSYKVILGAHQEVNLEPHVQEIEVSRLFLEPTRKDIALLKLSSPAVITDKVIPACLPSPNYVVADRTECFITGWGETQGTFGAGLLKEAQLPVIENKVCNRYEFLNGRVQSTELCAGHLAGGTDSCQGDAGGPLVCFEKDKYILQGVTSWGLGCARPNKPGVYVRVSRFVTWIEGVMRNN

(19) United States
(12) Patent Application Publication
(10) Pub. No.: US 2020/0140840 A1
(43) Pub. Date: May 7, 2020

(54) FACTOR IX VARIANTS AND METHODS OF USE THEREFOR

(71) Applicant: Wisconsin Alumni Research Foundation, Madison, WI (US)

(72) Inventors: John P. SHEEHAN, Middleton, WI (US); Pansakorn TANRATANA, Madison, WI (US)

(73) Assignee: Wisconsin Alumni Research Foundation, Madison, WI (US)

(21) Appl. No.: 16/685,129

(22) Filed: Nov. 15, 2019

Related U.S. Application Data
(62) Division of application No. 14/928,689, filed on Oct. 30, 2015, now abandoned.
(60) Provisional application No. 62/073,372, filed on Oct. 31, 2014.

(51) Int. Cl. C12N 9/64; A61K 38/48

(52) U.S. Cl. CPC C12N 9/644; A61K 38/4846; C12Y 304/21022

(57) ABSTRACT

Modified Factor IX (FIX) polypeptides, nucleic acid encoding the same, and methods of generating modified Factor IX polypeptides are provided. Also provided are pharmaceutical compositions that contain the modified Factor IX polypeptides, methods of treatment using modified Factor IX polypeptides, and assay for Factor IX activity.

This application is a divisional of U.S. application Ser. No. 14/928,689, filed Oct. 30, 2015, which claims benefit of priority to U.S. Provisional Application Ser. No. 62/073,372, filed Oct. 31, 2014, the entire contents of each of which are hereby incorporated by reference.

### STATEMENT OF FEDERAL FUNDING

This invention was made with government support under HL080452 awarded by the National Institutes of Health. The government has certain rights in the invention.

### INCORPORATION OF SEQUENCE LISTING

The sequence listing that is contained in the file named “WARFP0050USD1.txt”, which is 71 KB (as measured in Microsoft Windows®) and was created on Nov. 15, 2019, is filed herewith by electronic submission and is incorporated by reference herein.

### BACKGROUND OF THE INVENTION

### 1. Field

The present disclosure relates generally to the fields of biology and medicine. More particularly, it concerns variants of Factor IX which can be used in improved methods of treating hemophilia.

### 2. Description of Related Art

Hemophilia B is an X-linked bleeding disorder characterized by a deficiency of coagulation factor IX (Factor IX). Prophylactic infusion of replacement factor 2-3 times per week is superior to on demand therapy for preventing clinical complications in severe hemophilia patients (<1% plasma Factor VIII (FVIII) or Factor IX levels) (Manco-Johnson, 2003; Manco-Johnson et al., 2007). The goal of prophylaxis in hemophilia B is to maintain plasma Factor IX levels >1%, which has led to strategies designed to prolong the terminal plasma half-life of Factor IX. These strategies include linking recombinant Factor IX (rFactor IX) to albumin or the human Fc domain to facilitate uptake by the neonatal Fc receptor or PEGylation of Factor IX (Ostergaard et al., 2011; Martinowitz et al., 2013; Powell et al., 2013). However, the mechanisms for distribution and clearance of circulating Factor IX are incompletely understood. The apparent volume of distribution for Factor IX is significantly larger than plasma, characterized by a two-compartment pharmacokinetic model (Bjorjkman et al., 1994). These findings suggest a non-circulating “pool” of Factor IX bound to the vasculature and/or extravascular sites. Factor IX binds rapidly and reversibly to vascular endothelium and extracellular matrix, in part mediated by the interaction of specific residues in the Gla domain with collagen IV (Stern et al., 1983; 1987; Cheung et al., 1996). Mutagenesis of Factor IX demonstrates that reduced affinity for collagen IV results in a modest bleeding disorder, while enhanced affinity extends the therapeutic efficacy of rFactor IX well beyond when plasma levels fall below 1% (Gui et al., 2009; Feng et al., 2013). Thus, the extravascular pool plays an important role in the hemostatic function of Factor IX.

Factor X activation by the intrinsic tenase complex (Factor IXa-Factor VIIIa) is the rate-limiting step for thrombin generation (Rand et al., 1996). Consistent with its rate-limiting role in blood coagulation, Factor IXa (Factor IXa) is a highly regulated enzyme. The isolated protease ispoorly reactive with both substrates and inhibitors (Brandstetter et al., 1995; Hopfner et al., 1999), but exhibits a remarkable 10⁶-fold enhancement in catalytic activity within the intrinsic tenase complex, resulting from cofactor and substrate induced alterations in Factor IXa (Duffy et al., 1992; Zogg et al., 2009). The catalytic activity of Factor IXa is limited by cofactor instability (loss of A2 domain) (Fay et al., 1996) and inhibited by antithrombin (Fuchs et al., 1984). Antithrombin is the primary plasma inhibitor of Factor IXa and prominently localizes to anticoagulantly active heparan sulfate in the subendothelial basement membrane (de Agostini et al., 1990). Similar to other coagulation proteases, the regulation of Factor IXa involves the interaction of substrate, cofactor and inhibitors with protease exosites (Krishnaswamy, S., 2005). The Factor IXa protease domain contains a heparin/cofactor-binding exosite located near the C-terminal α-helix (Misenheimer et al., 2007; Yuan et al., 2005; Yang et al., 2002) and an antithrombin/substrate-binding exosite, in part, consisting of the autolysis loop (c143-154,) (Yang et al., 2003), (Factor IX residues are identified by chymotrypsinogen numbering system throughout this disclosure; see FIG. 7 for reference). The heparin-binding exosite participates in inhibition by the antithrombin-heparin complex, antithrombin-independent inhibition of the intrinsic tenase complex by heparin oligosaccharides, and critical cofactor interactions (Yuan et al., 2005; Yang et al., 2002; Sheehan et al., 2003). The antithrombin-binding exosite participates in a critical protein-protein interaction for acceleration of inhibition by the antithrombin-pentasaccharide complex, while neighboring residues contribute to a substrate-binding site on Factor IXa (Yang et al., 2003; Bested et al., 2003).

Mutagenesis of the Factor IXa protease domain demonstrates that the cofactor-binding site overlaps extensively with the heparin-binding site (Misenheimer et al., 2007; Yuan et al., 2005). Alanine substitutions that have the greatest effect on heparin binding (R165A and R233A) also substantially reduce cofactor affinity (FIG. 1) (Misenheimer and Sheehan, 2010). However, replacement of residues peripheral to the center of the heparin-binding site (K126A, K132A) reduces heparin affinity with only modest effects on protease-cofactor affinity (Misenheimer et al., 2007). Likewise, alanine substitution at R150 in gla-domainless Factor IX substantially reduces the rate of inhibition by antithrombin, while preserving the Factor X interaction (Yang et al., 2003). The co-crystal structure of the Factor IXa EGF2-protease domains with pentasaccharide-activated antithrombin confirms the critical role of R150, which participates in 9 distinct intermolecular interactions (Johnson et al., 2010). Thus, selected rFactor IX mutations achieve a degree of dissociation between heparin and cofactor binding (K126A, K132A) and between antithrombin and Factor X binding (R150A), respectively.

### SUMMARY

Thus, in accordance with the present disclosure, there is provided a Factor IX protein comprising a R→A substitution and residue 150 of the native sequence and either or both (a) a K→A substitution at residue 126 of the native sequence or (b) a K→A substitution at residues 132 of the native sequence, as defined by the chymotrypsinogen numbering system for the protease domain. The protein may be full length uncleaved Factor IX. The protein may lack the signal sequence of full length Factor IX. The protein may be cleaved to Factor IXa. The protein may have the mutation profile of 150R→A/126K→A/132K→A, 150R→A/126K→A or 150R→A/132K→A. The protein sequence may comprise or consist of SEQ ID NO: 2, SEQ ID NO: 4 or SEQ ID NO: 6. The protein may consist of a naked polypeptide chain. The protein may be glycosylated, carboxylated, hydroxylated, sulfated, phosphorylated, albuminated, or conjugated to a polyethylene glycol (PEG) moiety. The protein may be a precursor polypeptide. The protein may comprise a signal peptide. The protein may comprise a propeptide. The protein may lack a propeptide. The protein may be a zymogen. The protein may be secreted. The protein may comprise a heavy chain and/or a light chain.

Similarly, there is provided a Factor IX nucleid acid encoding a modified Factor IX protein comprising a R→A substitution and residue 150 of the native sequence and either or both (a) a K→A substitution at residue 126 of the native sequence or (b) a K→A substitution at residues 132 of the native sequence, as defined by the chymotrypsinogen numbering system for the protease domain. The nucleic acid may encode full length uncleaved Factor IX. The nucleic acid may encode a protein having the mutation profile of 150R→A/126K→A/132K→A, 150R→A/126K→A or 150R→A/132K→A. The nucleic acid may encode a protein comprising or consisting of SEQ ID NO: 2, SEQ ID NO: 4 or SEQ ID NO: 6. The nucleic acid may encode a precursor polypeptide. The nucleic acid may encode a protein comprising a signal peptide. The nucleic acid may encode a propeptide. The nucleic acid may encode a zymogen.

In another embodiment, there is provided a method of treating hemophilia or hemorrhagic disease comprising administering, to a subject in need thereof, a Factor IX protein comprising a R→A substitution at residue 150 of the native sequence and either or both (a) a K→A substitution at residue 126 of the native sequence or (b) a K→A substitution at residues 132 of the native sequence, as defined by the chymotrypsinogen numbering system for the protease domain. The protein may be full length uncleaved Factor IX. The protein may lack the signal sequence of full length Factor IX. The protein may be cleaved to Factor IXa. The protein may have the mutation profile of 150R→A/126K→A/132K→A, 150R→A/126K→A or 150R→A/132K→A. The protein sequence may comprise or consist of SEQ ID NO: 2, SEQ ID NO: 4 or SEQ ID NO: 6. The protein may consists of a naked polypeptide chain. The protein may be glycosylated, carboxylated, hydroxylated, sulfated, phosphorylated, albuminated, or conjugated to a polyethylene glycol (PEG) moiety. The protein may be a precursor polypeptide. The protein may comprise a signal peptide. The protein may comprise a propeptide. The protein may lack a propeptide. The protein may be a zymogen. The protein may be secreted. The protein may comprise a heavy chain and/or a light chain.

Administering may comprise intravenous delivery, subcutaneous delivery, or transdermal delivery. Subcutaneous delivery may comprise delivery through a pump or implantable depot device. The protein may be formulated with one or more of L-histidine, sucrose, glycine, and/or a polysorbate. The hemophilia may be hemophilia B. The hemophilia may be congenital or acquired.

In another embodiment, there is provided a kit comprising a Factor IX protein comprising a R→A substitution and residue 150 of the native sequence and either or both (a) a K→A substitution at residue 126 of the native sequence or (b) a K→A substitution at residues 132 of the native sequence, as defined by the chymotrypsinogen numbering system for the protease domain. The protein may be full length uncleaved Factor IX. The protein may lack the signal sequence of full length Factor IX. The protein may be cleaved to Factor IXa. The protein may have the mutation profile of 150R→A/126K→A/132K→A, 150R→A/126K→A or 150R→A/132K→A. The protein sequence may comprise or consist of SEQ ID NO: 2, SEQ ID NO: 4 or SEQ ID NO: 6. The kit may further comprise (a) a sterile buffer solution; (b) a device for administering said protein; and/or (c) instructions for performing administration.

In still a further embodiment, there is provided a method of detecting Factor IXa activity in a plasma test sample comprising (a) diluting said test sample in citrated Factor IX-deficient plasma; (b) incubating human Factor VIII with thrombin to produce activated Factor VIIIa; (c) neutralizing the thrombin of step (b) with hirudin; (d) adding the thrombin-activated Factor VIIIa of step (b) to the diluted test sample of step (a) immediately following recalcification and addition of a fluorogenic substrate of thrombin; (e) detecting plasma thrombin generation over time by cleavage of said fluorogenic substrate; and (f) comparing the thrombin cleavage of step (d) with a standard curve of Factor IXa and resulting cleavage of said fluorogenic substrate, wherein the amount or rate (peak thrombin or velocity index) of thrombin generation predicts the amount of Factor IXa in said sample.

Detecting may comprise use of a calibration curve constructed with α2-macroglobulin-thrombin complex to derive the thrombin concentration over time. The method may further comprise thrombin generation parameters selected from lag time, peak thrombin concentration, time to thrombin peak and velocity index are determined. The method may further comprise pre-incubation of test plasma with an inhibitory antibody to determine the specificity of thrombin generation. The inhibitory antibody may be an antibody that blocks Factor IX activation by Factor XIa, or an anti-Factor IX antibody that inhibits Factor IXa activity in plasma. The concentration of Factor VIII in step (a) may be about 19.2 nM; and/or the concentration of thrombin in step (a) may be about 12.8 nM; and/or the incubating in step (a) may be about 30 seconds; and/or the final plasma concentration of Factor VIIIa in step (c) may be about 1.3 nM; and/or the amount hirudin in step (b) may be about 1.25-fold molar excess of thrombin; and/or the fluorogenic substrate may be Z-Gly-Gly-Arg-AMC.

Still a further embodiment comprises a pharmaceutical composition, comprising the modified Factor IX polypeptide as described above. The pharmaceutical composition may be formulated for local, systemic or topical administration. The pharmaceutical formulation may be formulated for oral, nasal, pulmonary, buccal, transdermal, subcutaneous, intraduodenal, enteral, parenteral, intravenous, or intramuscular administration. The modified Factor IX polypeptide may be glycosylated, carboxylated, hydroxylated, sulfated, phosphorylated, albuminated, or conjugated to a polyethylene glycol (PEG) moiety or expressed as a fusion protein with a fusion partner that enhances half-life. The pharmaceutical composition may be formulated for administration as a liquid, a pill, a tablet, a lozenge and a capsule. The tablet or capsule may be enterically coated. The pharmaceutical composition may be formulated for controlled-release of the modified Factor IX polypeptide. The pill, tablet, lozenge, or capsule may deliver the modified Factor IX polypeptide to the mucosa of the mouth, throat, or gastrointestinal tract.

Also provided is an enhanced thrombin generation assay, or “ETGA,” which detects Factor IXa activity in test samples by dilution into citrated FIX-deficient plasma system. Briefly, a standard curve is established by adding test plasma containing human Factor IXa to Factor IX-deficient plasma. Simultaneously, human Factor VIII was activated with thrombin, neutralized with excess of hirudin, and the resulting thrombin-activated Factor Villa is added to plasma immediately after recalcification with the fluorogenic substrate. Plasma thrombin generation (TG) is detected by cleavage of fluorogenic substrate, and fluorescent data exported to evaluation software. Software generated TG parameters including lag time, peak thrombin concentration, time to thrombin peak and velocity index. Factor IXa concentration can be plotted versus mean peak thrombin±SEM (n=3) and the data fit to a parabolic function. Sample Factor IXa activity is obtained from the standard curve using mean peak thrombin concentration. The specificity of the TG response is determined by pre-incubation of test plasma with inhibitory antibodies. To block activity due to contact pathway-dependent Factor IXa generation during the assay, activity is determined in the presence of the monoclonal antibody which blocks Factor IX activation by Factor XIa. Similarly, the Factor IXa dependence of the activity is verified by pre-incubation test plasma with an inhibitory anti-Factor IX Gla domain antibody. An inhibitory anti-TF antibody had no effect on plasma activity in this assay.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. Representation of human FIXa crystal structure with mutations in the exosites highlighted. Representation of EGF2-protease hFIXa fragment (gray ribbon backbone) with the side chains of residues K126, K132, R165 and R233 in the heparin-binding exosite and R150 in the ATIII-binding exosite depicted as spheres (black). The active site serine (S195) is similarly depicted (light gray), as well as Ca+2 (medium gray). Structures were created in Pymol using PDB ID 1RFN with residues labeled using chymotrypsinogen numbering system.

FIGS. 2A-B. Coomassie stained gel and western blot of purified rFIX zymogens. (FIG. 2A) Purified pFIX and rFIX proteins (1 Vg) were run on a 10% SDS-PAGE gel (Next Gel), stained with GelCode Blue Safe Stain (for at least one hour) and destained in mill-Q water. (FIG. 2B) Purified pFIX or rFIX proteins (50 ng) were separated by 10% SDS-PAGE, transferred to PVD membrane and western blot performed with polyclonal donkey anti-human FIX-HRP conjugated antibody (1:10,000 dilution). Bands were visualized with Pierce® ECL 2 HRP Western Blot Substrate and x-ray film.

FIGS. 3A-F. Ability of rFIX(a) to support plasma thrombin generation in FIX-deficient plasma. Representative thrombin generation curves showing dose dependence in the presence of (FIG. 3A) 0, 1, 5, 10, 25 and 100% rFIX-WT and (FIG. 3B) 0, 20, 40 and 100 pM rFIXa-WT. Representative thrombin generation curves for selected recombinant proteins at (FIG. 3C) 100% rFIX (90 nM) and (FIG. 3D) 100 pM rFIX(a). Dose dependence of peak thrombinconcentration generated in the presence of (FIG. 3E) 0, 1, 5, 10, 25 and 100% (90 nM) plasma levels of rFIX and (FIG. 3F) 0, 20, 40 and 100 pM rFIXa. (n=3-4, ±SEM).

FIG. 4. Effect of antithrombin and heparin exosite mutations on the in vitro half-life of rFIXa in human FIX-deficient plasma. A standard curve was constructed using peak thrombin concentrations generated by FIX-deficient plasma samples supplemented with human pFIXa (0-80 pM) in the ETGA assay, as described in the Methods. Similarly, selected recombinant FIXa proteins (50-400 pM) in FIX-deficient plasma were incubated at 37° C. for 0-120 min prior to determination of residual FIXa activity in the ETGA assay. Peak thrombin concentration at each time point was converted to FIXa concentration using the standard curve. The FIXa activity over time was fit to the equation for first order decay A=A0e−kt, where A is activity, t is time, and k is the rate constant. Plasma half-life (t1/2) for each recombinant FIXa was expressed as the mean±SEM (n=3).

FIG. 5. Coomassie-stained gel of rFIXa proteases. Purified pFIXa and rFIXa proteins (1 μg) were run on a 10% Next Gel, stained with GelCode Blue Safe Stain (for at least one hour) and destained in Milli-Q water

FIGS. 6A-C. Representative antithrombin inhibition curves from Table 3 data. Inhibition of FIXa (292.5 nM) by (FIG. 6A) 4.5 μM ATIII, (FIG. 6B) 4.5 μM ATIII in the presence of 460 nM Fondaparinux, and (FIG. 6C) 4.5 μM ATIII in the presence of 0.24 U/mL UFH is presented for rFIXa WT, K126A, K132A, K126A/K132A, R150A, K126A/R150A, K132A/R150A and K126A/K132A/R150A and pFIXa (n=3-4, ±SEM).

FIG. 7. Comparison of the linear and chymotrypsinogen numbering systems. Excerpted from Bajaj and Birktoft (1993). (SEQ ID NOS: 9-31).

FIG. 8—Plasma factor IXa activity in volunteer blood donors. Factor IXa activity was determined in citrated platelet poor plasma from postmenopausal women not receiving hormone replacement therapy (Post-Men; N=36), premenopausal women not receiving oral contraceptives (Pre-Men, N=36), premenopausal women taking oral contraceptives (OCP, N=36) and males not on hormone therapy (Male; N=10). The factor IXa activity assay is described on pages 7 and 53 of this application and in our published work (Westmark, P. et al Journal of Thrombosis and Haemostasis June; 13:1053-63). Subject plasma (10 μl) was tested in factor IX deficient plasma in the presence of an anti-factor XIa inhibitory to block in situ factor IXa generation. Pre-incubation of subject samples with anti-factor IXa inhibitory antibody completely abrogated the activity in all cases. Addition of anti-tissue factor inhibitory antibody had no significant effect on measured activity. Median and quartiles are indicated with horizontal lines for each group. Data from OCP group was not normally distributed, thus groups were compared using Mann-Whitney. P-values were determined for: Men vs. Pre-Men (0.004), Post-Men (0.006) or OCP (<0.001); OCP vs. Pre-men (0.006) or Post-men (<0.001); and Pre-Men vs. Post-Men (not significant).

### DETAILED DESCRIPTION OF ILLUSTRATIVE EMBODIMENTS

As stated above, selected rFactor IX mutations achieve a reasonable degree of dissociation between heparin and cofactor binding (K126A, K132A) and between antithrombin and Factor X binding (R150A), respectively. The inventors hypothesized that selective disruption of exosite-mediated regulation of rFactor IXa by antithrombin and heparin/heparan sulfate would enhance the in vivo activity of rFactor IXa. Based on this hypothesis, rFactor IX(a) proteins with combined mutations in the heparin-binding and antithrombin-binding exosites were generated, expressed and characterized with regard to coagulant activity, plasma thrombin generation, inhibition by antithrombin and plasma half-life of rFactor IXa. The results show that selective mutagenesis of rFactor IX can synergistically disrupt antithrombin and heparin-based regulation, preserve plasma thrombin generation and prolong the plasma half-life of rFactor IXa. These and other aspects of the disclosure are discussed below.

### I. FACTOR IX

Factor IX (or Christmas factor) is one of the serine proteases of the coagulation system; it belongs to peptidase family S1. Deficiency of this protein causes hemophilia B. It was discovered in 1952 after a young boy named Stephen Christmas was found to be lacking this exact factor, leading to hemophilia.

Factor IX expression increases with age in both humans and mice. In mouse models mutations within the promoter region of Factor IX have an age-dependent phenotype. Factors VII, IX, and X all play key roles in blood coagulation and also share a common domain architecture. The Factor IX protein is composed of four protein domains: the Gla domain, two tandem copies of the EGF domain and a C-terminal trypsin-like peptidase domain which carries out the catalytic cleavage.

Deficiency of Factor IX causes Christmas disease (hemophilia B). Over 100 mutations of factor IX have been described; some cause no symptoms, but many lead to a significant bleeding disorder. The original Christmas disease mutation was identified by sequencing of Christmas' DNA, revealing a mutation which changed a cysteine to a serine. Some rare mutations of factor IX result in elevated clotting activity, and can result in clotting diseases, such as deep vein thrombosis. Factor IX deficiency is generally treated by injection of purified factor IX produced through cloning in various animal or animal cell vectors.

Factor IX is a vitamin K-dependent serine protease and is an important coagulation factor in hemostasis. It is synthesized as a single chain zymogen in the liver and circulates in the blood in this inactivated state until activated as part of the coagulation cascade. Following activation from the Factor IX zymogen to activated Factor IXa (FIXa) by Factor XIa or the TF/Factor VIIa complex, Factor IXa binds it's cofactor, Factor VIIIa. The resulting Factor IXa/Factor Villa complex binds and activates Factor X to Factor Xa, thus continuing the coagulation cascade described above to establish hemostasis. The concentration of Factor IX in the blood is approximately 4-5 μg/mL, and it has a half-life of approximately 18-24 hours.

### A. Factor IX Structure

The human Factor IX gene is located on the X chromosome and is approximately 34 kb long with eight exons. The human Factor IX transcript is 2803 nucleotides and contains a short 5′ untranslated region, an open reading frame (including stop codon) of 1383 nucleotides and a 3′ untranslated region. The 1383 nucleotide open reading frame encodes a 461 amino acid precursor polypeptide (Swiss-Prot accession no. P00740) containing a 28 amino acid N-terminal signal peptide (aa 1-28) that directs the Factor IX polypeptide to the cellular secretory pathway. In addition the hydrophobic signal peptide, the Factor IX precursor polypeptide also contains an 18 amino acid propeptide (aa 29-46) that, when cleaved, releases the 415 amino acid mature polypeptide that circulates in the blood as a zymogen until activation to Factor IXa. In addition to the signal peptide and propeptide, the Factor IX precursor also contains the following segments and domains: a Gla domain (aa 47-92, corresponding to aa 1-46 of the mature Factor IX protein), epidermal growth factor (EGF)-like domain 1 (EGF1; aa 93-129, corresponding to aa 47-83 of the mature Factor IX protein), EGF2 (aa 130-171, corresponding to aa 84-125 of mature Factor IX protein), a light chain (aa 47-191, corresponding to aa 1-145 of the mature Factor IX protein), an activation peptide (aa 192-226, corresponding to aa 146-180 of the mature Factor IX protein), a heavy chain (aa 227-461, corresponding to aa 181-415 of the mature Factor IX protein) and a serine protease domain (aa 227-459, corresponding to aa 181-413 of the mature Factor IX protein). The wild-type protein is shown at SEQ ID NO: 8, and the corresponding nucleic acid at SEQ ID NO: 7.

Like other vitamin K-dependent proteins, such as prothrombin, coagulation factors VII and X, and proteins C, S, and Z, the Gla domain of Factor IX is a membrane binding motif which, in the presence of calcium ions, interacts with the phospholipid membranes of cells. The vitamin K-dependent proteins require vitamin K for the posttranslational synthesis of γ-carboxyglutamic acid, an amino acid clustered in the Gla domain of these proteins. The Factor IX Gla domain has 12 glutamic residues, each of which is a potential carboxylation site. Many of them are, therefore, modified by carboxylation to generate γ-carboxyglutamic acid residues. There are a total of eight Ca²⁺ binding sites, of both high and low affinity, in the Factor IX Gla domain that, when occupied by calcium ions, facilitate correct folding of the Gla domain to expose hydrophobic residues in the Factor IX polypeptide that are inserted into the lipid bilayer to effect binding to the membrane.

In addition to the Gla domain, the Factor IX polypeptide also contains two EGF-like domains. Each EGF-like domain contains six highly conserved cysteine residues that form three disulphide bonds in each domain in the same pattern observed in the EGF protein. The first EGF-like domain (EGF1) is a calcium-binding EGF domain containing a high affinity Ca²⁺ binding site that, when occupied by a calcium ion, contributes to the correct folding of the molecule and promotes biological activity. The second EGF domain (EGF2) does not contain a calcium binding site.

The serine protease domain, or catalytic domain, of Factor IX is the domain responsible for the proteolytic activity of Factor IXa. Like other serine proteases, Factor IX contains a serine protease catalytic triad composed of H221, D269 and S365 (corresponding to H57, D102 and S195 by chymotrypsin numbering).

Activation of mature Factor IX to Factor IXa is effected by proteolytic cleavage of the R145-A146 bonds and R180-V181 bonds, releasing the activation peptide that corresponds to aa 146-180 of the mature Factor IX protein. Thus, following activation, Factor IXa consists of two chains; the light chain and heavy chain. The light chain contains the Gla domain, EGF1 and EGF2 domains, and the heavy chain contains the protease domain. The two chains are held together by a single disulphide bond between C132 and C289.

### B. Factor IX Post-Translational Modification

The Factor IX precursor polypeptide undergoes extensive posttranslational modification to become the mature zymogen that is secreted into the blood. Such posttranslation modifications include γ-carboxylation, β-hydroxylation, cleavage of the signal peptide and propeptide, O- and N-linked glycosylation, sulfation and phosphorylation. The N-terminal signal peptide directs the polypeptide to the endoplasmic reticulum (ER), after which it is cleaved. Immediately prior to secretion from the cell, the propeptide is cleaved by processing proteases, such as, for example, PACE/furin, that recognize at least two arginine residues within four amino acids prior to the cleavage site.

A single enzyme, vitamin K-dependent gamma-carboxylase, catalyzes the γ-carboxylation Factor IX in the ER. In the carboxylation reaction, the γ-carboxylase binds to the Factor IX propeptide and catalyzes a second carboxylation on the -carbon of the glutamic acid residues (i.e., Glu to γ-carboxyglutamyl or Gla) in the Gla domain of the polypeptide. Assuming all glutamic acid residues are γ-carboxylated, Factor IX contains 12 Gla residues, where the first 10 are at homologous positions of other vitamin K-dependent proteins. The γ-carboxylase tends to processively carboxylate all glutamates in the Gla domain of Factor IX before releasing the substrate.

Factor IX also is partially β-hydroxylated. This modification is performed by a dioxygenase, which hydroxylates the β-carbon of D64 in EGF1. Approximately one third of human Factor IX polypeptides are β-hydroxylated. Although D64 contributes to the high affinity Ca²⁺ binding site in the EGF1 domain of Factor IX, the hydroxylation of this residue does not appear to be necessary for Ca²⁺ binding, nor for biological activity. Additional post-translational modifications include sulfonation at the tyrosine at position 155, and phosphorylation at the serine residue at position 158. These post-translational modifications of Factor IX have been implicated in contributing to in vivo recovery of Factor IX (U.S. Pat. No. 7,575,897).

Factor IX is N-linked glycosylated at asparagine residues in the activation peptide corresponding to N157 and N167. Post-translational modification also results in the serine residue at position 53 having O-linked disaccharides and trisaccharides, while the serine residue at position 61 contains an O-linked tetrasaccharide. Additionally, the threonine residues at amino acid positions 159 and 169 are O-glycosylated. The threonine residues at amino acid positions 172 and 179 also may be O-glycosylated.

### C. Factor IX Activation

Factor IX circulates predominantly as a zymogen with minimal proteolytic activity until it is activated by proteolytic cleavage. Activation can be effected by the TF/FVIIa complex or Factor XIa. Activation by TF/FVIIa is through the intrinsic pathway, while activation by Factor XIa is through the extrinsic pathway, described above. The process of activation appears to be sequential with initial cleavage of the Arg145-Ala146 bond, followed by cleavage of the Arg180-Vail 81 bond. The proteolytic cleavage releases the activation peptide, forming the two-chain Factor IXa molecule containing the light chain (corresponding to amino acid positions 1-145) and heavy chain (corresponding to amino acid positions 181-415) held together by a disulphide bond between the two cysteines at amino acid positions 132 and 289 (numbering corresponding to the mature Factor IX polypeptide).

At least two exosites in Factor X appear to be involved in binding to TF in the TF/FVIIa complex to form the Factor IX/TF/Factor VIIa ternary complex. Studies suggest that the EGF1 domain of Factor IX is required for Factor IX activation by the TF/Factor VIIa complex. For example, mutation of G48 (relative to the mature Factor IX polypeptide) in the EGF1 domain of Factor IX reduces its activation by TF/Factor VIIa. Further, the EGF1 domain of Factor IX has been shown to interact with TF in the TF/Factor VIIa complex. In contrast, however, the EGF1 domain does not appear to be required for Factor IX activation by Factor XIa. The Gla domain also is involved in binding to the TF/Factor VIIa complex and, therefore, in activation. The Gla domain of Factor IX interacts with the same region in TF as Factor X, which also is activated by the TF/FVIIa complex.

Following cleavage and release of the activation peptide, a new amino terminus at V181 (corresponding to the mature Factor IX polypeptide; V16 by chymotrypsin numbering) is generated. Release of the activation peptide facilitates a conformational change whereby the amino group of V181 inserts into the active site and forms a salt bridge with the side chain carboxylate of D364. Such a change is required for conversion of the zymogen state to an active state, as the change converts the hydroxyl side chain of S365 to a reactive species that is able to hydrolyze the cleavage site of its substrate, Factor X. The activated Factor IXa polypeptide remains in a zymogen-like conformation until additional conformational changes are induced, such as by binding with Factor VIIIa, to generate a Factor IXa polypeptide with maximal catalytic activity.

### D. Factor IX Function

Factor IX plays an important role in the coagulation pathway and a deficiency or absence of Factor IX activity leads to hemophilia B. Once activated from Factor IX to Factor IXa, Factor IXa in turn functions to activate the large amounts of Factor X to Factor Xa that are required for coagulation. To do so, Factor IXa must first bind to its cofactor, Factor VIIIa, to form the Factor IXa/Factor VIIIa complex, also called the intrinsic tenase complex, on the phospholipid surface of the activated platelet. Both the Gla domain and EGF2 domain of Factor IX are important for stable binding to phospholipids. The Factor IXa/Factor VIIIa complex then binds Factor X to cleave this coagulation factor to form Factor IXa.

Factor IXa is virtually inactive in the absence of its cofactor, Factor VIIIa, and physiologic substrate, Factor X. Experimental studies indicate that this can be attributed mainly to the 99-loop. When Factor IXa is not bound by its cofactor, Y177 locks the 99-loop in an inactive conformation in which the side chains of Y99 and K98 (by chymotrypsin numbering, corresponding to Y266 and K265 of the mature Factor IX polypeptide) impede substrate binding. Binding of Factor VIIIa to Factor IXa unlocks and releases this zymogen-like conformation, and Factor X is then able to associate with the Factor IXa/Factor VIIIa complex and rearrange the unlocked 99-loop, subsequently binding to the active site cleft. The binding of Factor IXa to phospholipids and the presence of Ca²⁺ further enhances the reaction.

Several models of the Factor IXa/Factor VIIIa interaction have been proposed. Factor IXa binds to Factor VIIIa in an interaction involving more than one domain of the Factor IXa polypeptide. Factor VIIIa is a heterodimer composed of three noncovalently associated chains: A1, A2 and A3-C₁-C₂. A3-C₁-C₂ also is referred to as the light chain. The protease domain of Factor IXa appears to interact with the A2 subunit of Factor VIIIa. Studies suggest that the 293-helix (126-helix by chymotrypsin numbering), 330-helix (162-helix by chymotrypsin numbering) and N346 (N178) by chymotrypsin numbering) of Factor IXa are involved in the interaction with the A2 subunit of Factor VIIIa. The EGF1/EGF2 domains of Factor IXa interact with the A3 subunit of Factor VIIIa. Further, it is postulated that the Gla domain of Factor IXa interacts with the C2 domain of Factor VIIIa. Calcium ions and phospholipids also contribute to binding of Factor IXa and Factor VIIIa. For example, the presence of phospholipids increases the binding of Factor IXa to Factor VIIIa by approximately 2000-fold. Following binding of Factor X by the Factor IXa/Factor VIIIa complex, the protease domain (or catalytic domain) of Factor IXa is responsible for cleavage of Factor X at R194-1195 to form Factor Xa.

The activity of Factor IXa is regulated by inhibitory molecules, such as the AT-III/heparin complex, as discussed above, and other clearance mechanisms, such as the low-density lipoprotein receptor-related protein (LRP). LRP is a membrane glycoprotein that is expressed on a variety of tissues, including liver, brain, placenta and lung. LRP binds a wide range of proteins and complexes in addition to Factor IXa, including, but not limited to, apolipoproteins, lipases, proteinases, proteinase-inhibitor complexes, and matrix proteins. The zymogen or inactive form of Factor IX does not bind LRP. Rather, upon activation, an LRP-binding site is exposed. This binding site is located in a loop in the protease domain spanning residues 342 to 346 of the mature Factor IX polypeptide.

### E. Factor IX as a Biopharmaceutical

Factor IX is integrally involved in the blood coagulation process, where, in its activated form (Factor IXa), it forms a tenase complex with Factor VIIIa and activates Factor X to Factor Xa. Factor Xa, in conjunction with phospholipids, calcium and Factor Va, converts prothrombin to thrombin, which in turn cleaves fibrinogen to fibrin monomers, thus facilitating the formation of a rigid mesh clot. Many studies have demonstrated the ability of exogenous Factor IX to promote blood clotting in patients with hemophilia. For example, hemophilia B patients, who are deficient in Factor IX, can be treated by replacement therapy with exogenous Factor IX. Early replacement therapies utilized plasma purified Factor IX, such as therapeutics marketed as MonoNine® Factor IX and Alpha-nine-SD® Factor IX. Plasma purified Factor IX complex therapeutics also have been used, including Bebulin® VH, a purified concentrate of Factor IX with Factor X and low amounts of Factor VII; Konyne® 80 (Bayer), a purified concentrate of Factor IX, with Factor II, Factor X, and low levels of Factor VII; PROPLEX® T (Baxter International), a heat treated product prepared from pooled normal human plasma containing Factor IX with Factor II, Factor VII, and Factor X; and Profilnine SD® (Alpha Therapeutic Corporation). More recently, however, a human recombinant Factor IX (BeneFIX® Coagulation Factor IX (Recombinant), Wyeth) has been approved for use in the control and prevention of bleeding episodes in hemophilia B patients, including control and prevention of bleeding in surgical settings. BeneFIX® Coagulation Factor IX (Recombinant) is identical to the Ala148 allelic form of plasma-derived Factor IX. Thus, compared to the wild-type Factor IX polypeptide, BeneFIX®, Coagulation Factor IX (Recombinant) contains a T148A mutation.

In addition to its use as a procoagulant, inactive forms of Factopr IX, or forms with reduced catalytic activity, can be used as an anticoagulant, such as in the treatment of thrombotic diseases and conditions. Typically, Factor IX is administered intravenously, but also can be administered orally, systemically, buccally, transdermally, intramuscularly and subcutaneously. Factor IX can be administered once or multiple times. Generally, multiple administrations are used in treatment regimens with Factor IX to effect coagulation.

### F. Definitions

As used herein, an “active portion or fragment of a factor IX polypeptide” refers to a portion of a human or non-human Factor IX polypeptide that includes at least one modification provided herein and exhibits an activity, such as one or more activities of a full-length Factor IX polypeptide or possesses another activity. Such activities include, but are not limited to peptidase activity, any coagulant (also referred to as procoagulant) activity, anticoagulant activity or other activity. Activity can be any percentage of activity (more or less) of the full-length polypeptide, including but not limited to, 1% of the activity, 2%, 3%, 4%, 5%, 10%, 20%, 30%, 40%, 50%, 60%, 70%, 80%, 90%, 95%, 96%, 97%, 98%, 99%, 100%, 200%, 300%, 400%, 500%, or more activity compared to the full polypeptide. Assays to determine function or activity of modified forms of FIX include those known to those of skill in the art, and exemplary assays are included herein. Assays include, for example, activated partial thromboplastin time (aPTT). In one such assay, coagulation activity is measured as the time required for formation of a fibrin clot. Activity also includes activities possessed by a fragment or modified form that are not possessed by the full-length polypeptide or unmodified polypeptide.

As used herein, “mature factor IX” refers to a Factor IX polypeptide that lacks a signal sequence and a propeptide sequence. Typically, a signal sequence targets a protein for secretion via the endoplasmic reticulum (ER)-golgi pathway and is cleaved following insertion into the ER during translation. A propeptide sequence typically functions in post-translational modification of the protein and is cleaved prior to secretion of the protein from the cell. Thus, a mature Factor IX polypeptide is typically a secreted protein.

As used herein, “native factor IX” refers to a Factor IX polypeptide encoded by a naturally occurring Factor IX gene that is present in an organism in nature, including a human or other animal. Included among native Factor IX polypeptides are the encoded precursor polypeptide, fragments thereof, and processed forms thereof, such as a mature form lacking the signal peptide as well as any pre- or post-translationally processed or modified form thereof.

As used herein, “activated Factor IX” or “FIXa” refers to a FIX polypeptide that has been proteolytically cleaved to activate the peptidase activity of the FIX polypeptide.

As used herein, a “zymogen” refers to any compound, such as a polypeptide, that is an inactive precursor of an enzyme and requires some change, such as proteolysis of the polypeptide, to become active. For example, FIX polypeptides exist in the blood plasma as zymogens until activation of the coagulation cascade, whereby the FIX polypeptides are cleaved by activated FXI.

### II. HEMOPHILIA B

Haemophilia B (or hemophilia B) is a blood clotting disorder caused by a mutation of the Factor IX gene, leading to a deficiency of Factor IX. It is the second-most common form of hemophilia. Factor IX is an X-linked recessive trait, which explains why, as in hemophilia A, usually only males are affected. One in 20,000-30,000 males are affected. While less prevalent than Hemophilia A, Hemophilia B remains a significant disease in which recurrent joint bleeds can lead to synovial hypertrophy, chronic synovitis, with destruction of synovium, cartilage, and bone leading to chronic pain, stiffness of the joints, and limitation of movement because of progressive severe joint damage. Recurrent muscle bleeds also produce acute pain, swelling, and limitation of movement, while bleeding at other sites can contribute to morbidity and mortality.

In 1990, George Brownlee and Merlin Crossley showed that two sets of genetic mutations were preventing two key proteins from attaching to the DNA of people with a rare and unusual form of hemophilia B—hemophilia B Leyden—where sufferers experience episodes of excessive bleeding in childhood but have few bleeding problems after puberty. This lack of protein attachment to the DNA was thereby turning off the gene that produces clotting factor IX, which prevents excessive bleeding. In 2013, Merlin Crossley discovered the third and final protein causing haemophilia B Leyden.

Treatment (bleeding prophylaxis) is by intravenous infusion of purified or recombinant Factor IX, as well as modified versions thereof (commercially available as BeneFIX and Alprolix). Factor IX has a longer half life than Factor VIII (deficient in haemophilia A) and as such Factor IX can be transfused less frequently. Tranexamic acid may be of value in patients undergoing surgery who have inherited Factor IX deficiency in order to reduce the perioperative risk of bleeding.

### III. MODIFIED FACTOR IX PROTEINS

A. Structure-Function Relationship

The activity of Factor IX(a) is regulated by heparin/heparin sulfate and antithrombin, which interact with separate exosites on the protease. The inventors' laboratory has demonstrated via mutagenesis of the Factor IXa protease domain that the cofactor (Factor VIIIa)-binding site overlaps extensively with the heparin-binding site. Alanine substitutions that have the greatest effect on heparin binding (R165A and R233A) also substantially reduce cofactor affinity (Factor IX residues are identified by the chymotrypsinogen numbering system) and coagulant activity. However, replacement of residues peripheral to the center of the heparin-binding site (K126A, K132A) reduces heparin affinity with only modest effects on protease-cofactor affinity. Likewise, alanine substitution at R150 substantially reduces the rate of inhibition by antithrombin, while preserving the substrate interaction with Factor X. The co-crystal structure of the Factor IXa EGF2-protease domains with pentasaccharide-activated antithrombin confirms the critical role of R150, which participates in 9 distinct intermolecular interactions. Thus, selected rFactor IX mutations achieve a reasonable degree of dissociation between heparin and cofactor binding (K126A, K132A) and between antithrombin and Factor X binding (R150A), respectively.

The inventors hypothesized that selective disruption of exosite-mediated regulation by antithrombin and heparin/heparan sulfate would enhance the in vivo activity of recombinant Factor IXa (rFIXa). Based on this hypothesis, rFIX(a) proteins with combined mutations in the heparin-binding and antithrombin-binding exosites were expressed and characterized with regard to coagulant activity, plasma thrombin generation, inhibition by antithrombin and plasma half-life of rFIXa. Single or combined (K126A/R150A or K132A/R150A) exosite mutations variably reduced coagulant activity relative to wild-type (WT) for FIX (27-91%) and FIXa (25-91%). Double mutation in the heparin exosite (K126A/K132A and K126A/K132A/R150A) markedly reduced coagulant activity (7-21%) and plasma TG. In contrast to coagulant activity, FIX K126A (1.8-fold), R150 (1.6-fold) and K132A/R150A (1.3-fold) supported increased tissue factor initiated plasma TG; while FIX K132A and K126A/R150A were similar to WT. FIXa K126A/R150A and K132A/R150A (1.5-fold) demonstrated significantly increased FIXa-initiated TG; while FIXa K132A, R150A and K126A (0.8-0.9 fold) were similar to WT. Dual mutations in the heparin exosite or combined mutations in both exosites synergistically reduced the inhibition rate for antithrombin-heparin. The half-life of FIXa WT in FIX-deficient plasma was remarkably lengthy (40.9±1.4 min) and further prolonged for FIXa R150A, K126A/R150A and 132A/R150A (>2 hr).

B. Production

Factor IX polypeptides, including modified Factor IX polypeptides, or domains thereof, of Factor IX can be obtained by methods well known in the art for protein purification and recombinant protein expression. Any method known to those of skill in the art for identification of nucleic acids that encode desired genes can be used. Any method available in the art can be used to obtain a full length (i.e., encompassing the entire coding region) cDNA or genomic DNA clone encoding a Factor IX polypeptide or other vitamin-K polypeptide, such as from a cell or tissue source, such as for example from liver. Modified Factor IX polypeptides can be engineered as described herein, such as by site-directed mutagenesis.

Factor IX can be cloned or isolated using any available methods known in the art for cloning and isolating nucleic acid molecules. Such methods include PCR amplification of nucleic acids and screening of libraries, including nucleic acid hybridization screening, antibody-based screening and activity-based screening.

Methods for amplification of nucleic acids can be used to isolate nucleic acid molecules encoding a Factor IX polypeptide, including for example, polymerase chain reaction (PCR) methods. A nucleic acid containing material can be used as a starting material from which a Factor IX-encoding nucleic acid molecule can be isolated. For example, DNA and mRNA preparations, cell extracts, tissue extracts (e.g., from liver), fluid samples (e.g., blood, serum, saliva), samples from healthy and/or diseased subjects can be used in amplification methods. Nucleic acid libraries also can be used as a source of starting material. Primers can be designed to amplify a Factor IX-encoding molecule. For example, primers can be designed based on expressed sequences from which a Factor IX is generated. Primers can be designed based on back-translation of a Factor IX amino acid sequence. Nucleic acid molecules generated by amplification can be sequenced and confirmed to encode a Factor IX polypeptide.

Additional nucleotide sequences can be joined to a Factor IX-encoding nucleic acid molecule, including linker sequences containing restriction endonuclease sites for the purpose of cloning the synthetic gene into a vector, for example, a protein expression vector or a vector designed for the amplification of the core protein coding DNA sequences. Furthermore, additional nucleotide sequences specifying functional DNA elements can be operatively linked to a Factor IX-encoding nucleic acid molecule. Examples of such sequences include, but are not limited to, promoter sequences designed to facilitate intracellular protein expression, and secretion sequences designed to facilitate protein secretion. Additional nucleotide sequences such as sequences specifying protein binding regions also can be linked to Factor IX-encoding nucleic acid molecules. Such regions include, but are not limited to, sequences to facilitate uptake of Factor IX into specific target cells, or otherwise enhance the pharmacokinetics of the synthetic gene.

The identified and isolated nucleic acids can then be inserted into an appropriate cloning vector. A large number of vector-host systems known in the art can be used. Possible vectors include, but are not limited to, plasmids or modified viruses, but the vector system must be compatible with the host cell used. Such vectors include, but are not limited to, bacteriophages such as lambda derivatives, or plasmids such as pBR322 or pUC plasmid derivatives or the Bluescript vector (Stratagene, La Jolla, Calif.). The insertion into a cloning vector can, for example, be accomplished by ligating the DNA fragment into a cloning vector which has complementary cohesive termini. Insertion can be effected using TOPO cloning vectors (Invitrogen, Carlsbad, Calif.). If the complementary restriction sites used to fragment the DNA are not present in the cloning vector, the ends of the DNA molecules can be enzymatically modified. Alternatively, any site desired can be produced by ligating nucleotide sequences (linkers) onto the DNA termini; these ligated linkers can contain specific chemically synthesized oligonucleotides encoding restriction endonuclease recognition sequences. In an alternative method, the cleaved vector and Factor IX protein gene can be modified by homopolymeric tailing. Recombinant molecules can be introduced into host cells via, for example, transformation, transfection, infection, electroporation and sonoporation, so that many copies of the gene sequence are generated.

In specific embodiments, transformation of host cells with recombinant DNA molecules that incorporate the isolated Factor IX protein gene, cDNA, or synthesized DNA sequence enables generation of multiple copies of the gene. Thus, the gene can be obtained in large quantities by growing transformants, isolating the recombinant DNA molecules from the transformants and, when necessary, retrieving the inserted gene from the isolated recombinant DNA.

1. Vectors and Cells

For recombinant expression of one or more of the Factor IX proteins, the nucleic acid containing all or a portion of the nucleotide sequence encoding the Factor IX protein can be inserted into an appropriate expression vector, i.e., a vector that contains the necessary elements for the transcription and translation of the inserted protein coding sequence. Exemplary of such a vector is any mammalian expression vector such as, for example, pCMV. The necessary transcriptional and translational signals also can be supplied by the native promoter for Factor IX genes, and/or their flanking regions.

Also provided are vectors that contain nucleic acid encoding the Factor IX or modified Factor IX. Cells containing the vectors also are provided. The cells include eukaryotic and prokaryotic cells, and the vectors are any suitable for use therein.

Prokaryotic and eukaryotic cells, including endothelial cells, containing the vectors are provided. Such cells include bacterial cells, yeast cells, fungal cells, Archea, plant cells, insect cells and animal cells. The cells are used to produce a Factor IX polypeptide or modified Factor IX polypeptide thereof by growing the above-described cells under conditions whereby the encoded Factor IX protein is expressed by the cell, and recovering the expressed Factor IX protein. For purposes herein, the Factor IX can be secreted into the medium.

In one embodiment, vectors containing a sequence of nucleotides that encodes a polypeptide that has Factor IX activity and contains all or a portion of the Factor IX polypeptide, or multiple copies thereof, are provided. The vectors can be selected for expression of the Factor IX polypeptide or modified Factor IX polypeptide thereof in the cell or such that the Factor IX protein is expressed as a secreted protein. When the Factor IX is expressed the nucleic acid is linked to nucleic acid encoding a secretion signal, such as the Saccharomyces cerevisiae α-mating factor signal sequence or a portion thereof, or the native signal sequence.

A variety of host-vector systems can be used to express the protein coding sequence. These include but are not limited to mammalian cell systems infected with virus (e.g., vaccinia virus, adenovirus and other viruses); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system used, any one of a number of suitable transcription and translation elements can be used.

Any methods known to those of skill in the art for the insertion of DNA fragments into a vector can be used to construct expression vectors containing a chimeric gene containing appropriate transcriptional/translational control signals and protein coding sequences. These methods can include in vitro recombinant DNA and synthetic techniques and in vivo recombinants (genetic recombination). Expression of nucleic acid sequences encoding a Factor IX polypeptide or modified Factor IX polypeptide, or domains, derivatives, fragments or homologs thereof, can be regulated by a second nucleic acid sequence so that the genes or fragments thereof are expressed in a host transformed with the recombinant DNA molecule(s). For example, expression of the proteins can be controlled by any promoter/enhancer known in the art. In a specific embodiment, the promoter is not native to the genes for a Factor IX protein. Promoters which can be used include but are not limited to the SV40 early promoter (Bemoist and Chambon, Nature 290:304-310 (1981)), the promoter contained in the 3′ long terminal repeat of Rous sarcoma virus (Yamamoto et al. Cell 22:787-797 (1980)), the herpes thymidine kinase promoter (Wagner et al., Proc. Natl. Acad. Sci. USA 78:1441-1445 (1981)), the regulatory sequences of the metallothionein gene (Minster et al., Nature 296:39-42 (1982)); prokaryotic expression vectors such as the β-lactamase promoter (Jay et al., (1981) Proc. Natl. Acad. Sci. USA 78:5543) or the tac promoter (DeBoer et al., Proc. Natl. Acad. Sci. USA 80:21-25 (1983)); see also “Useful Proteins from Recombinant Bacteria”: in Scientific American 242:79-94 (1980)); plant expression vectors containing the nopaline synthetase promoter (Herrara-Estrella et al., Nature 303:209-213 (1984)) or the cauliflower mosaic virus 35S RNA promoter (Garder et al., Nucleic Acids Res. 9:2871 (1981)), and the promoter of the photosynthetic enzyme ribulose bisphosphate carboxylase (Herrera-Estrella et al., Nature 310:115-120 (1984)); promoter elements from yeast and other fungi such as the Gal4 promoter, the alcohol dehydrogenase promoter, the phosphoglycerol kinase promoter, the alkaline phosphatase promoter, and the following animal transcriptional control regions that exhibit tissue specificity and have been used in transgenic animals: elastase I gene control region which is active in pancreatic acinar cells (Swift et al., Cell 38:639-646 (1984); Ornitz et al., Cold Spring Harbor Symp. Quant. Biol. 50:399-409 (1986); MacDonald, Hepatology 7:425-515 (1987)); insulin gene control region which is active in pancreatic beta cells (Hanahan et al., Nature 315:115-122 (1985)), immunoglobulin gene control region which is active in lymphoid cells (Grosschedl et al., Cell 38:647-658 (1984); Adams et al., Nature 318:533-538 (1985); Alexander et al., Mol. Cell. Biol. 7:1436-1444 (1987)), mouse mammary tumor virus control region which is active in testicular, breast, lymphoid and mast cells (Leder et al., Cell 45:485-495 (1986)), albumin gene control region which is active in liver (Pinkert et al., Genes and Devel. 1:268-276 (1987)), alpha-fetoprotein gene control region which is active in liver (Krumlauf et al., Mol. Cell. Biol. 5:1639-1648 (1985); Hammer et al., Science 235:53-58 1987)), alpha-1 antitrypsin gene control region which is active in liver (Kelsey et al., Genes and Devel. 1:161-171 (1987)), beta globin gene control region which is active in myeloid cells (Magram et al., Nature 315:338-340 (1985); Kollias et al., Cell 46:89-94 (1986)), myelin basic protein gene control region which is active in oligodendrocyte cells of the brain (Readhead et al., Cell 48:703-712 (1987)), myosin light chain-2 gene control region which is active in skeletal muscle (Shani, Nature 314:283-286 (1985)), and gonadotrophic releasing hormone gene control region which is active in gonadotrophs of the hypothalamus (Mason et al., Science 234:1372-1378 (1986)).

In a specific embodiment, a vector is used that contains a promoter operably linked to nucleic acids encoding a Factor IX polypeptide or modified Factor IX polypeptide, or a domain, fragment, derivative or homolog, thereof, one or more origins of replication, and optionally, one or more selectable markers (e.g., an antibiotic resistance gene). Vectors and systems for expression of Factor IX polypeptides include the well known Pichia vectors (available, for example, from Invitrogen, San Diego, Calif.), particularly those designed for secretion of the encoded proteins. Exemplary plasmid vectors for expression in mammalian cells include, for example, pCMV. Exemplary plasmid vectors for transformation of E. coli cells, include, for example, the pQE expression vectors (available from Qiagen, Valencia, Calif.; see also literature published by Qiagen describing the system). pQE vectors have a phage T5 promoter (recognized by E. coli RNA polymerase) and a double lac operator repression module to provide tightly regulated, high-level expression of recombinant proteins in E. coli, a synthetic ribosomal binding site (RBS II) for efficient translation, a 6×His tag coding sequence, to and T1 transcriptional terminators, ColE1 origin of replication, and a beta-lactamase gene for conferring ampicillin resistance. The pQE vectors enable placement of a 6.times.His tag at either the N- or C-terminus of the recombinant protein. Such plasmids include pQE 32, pQE 30, and pQE 31 which provide multiple cloning sites for all three reading frames and provide for the expression of N-terminally 6×His-tagged proteins. Other exemplary plasmid vectors for transformation of E. coli cells, include, for example, the pET expression vectors (see, U.S. Pat. No. 4,952,496; available from NOVAGEN, Madison, Wis.; see, also literature published by Novagen describing the system). Such plasmids include pET 11a, which contains the T7 lac promoter, T7 terminator, the inducible E. coli lac operator, and the lac repressor gene; pET 12a-c, which contains the T7 promoter, T7 terminator, and the E. coli ompT secretion signal; and pET 15b and pET19b (NOVAGEN, Madison, Wis.), which contain a His-Tag® leader sequence for use in purification with a His column and a thrombin cleavage site that permits cleavage following purification over the column, the T7-lac promoter region and the T7 terminator.

2. Expression Systems

Factor IX polypeptides (modified and unmodified) can be produced by any methods known in the art for protein production including in vitro and in vivo methods such as, for example, the introduction of nucleic acid molecules encoding Factor IX into a host cell, host animal and expression from nucleic acid molecules encoding Factor IX in vitro. Factor IX and modified Factor IX polypeptides can be expressed in any organism suitable to produce the required amounts and forms of a Factor IX polypeptide needed for administration and treatment. Expression hosts include prokaryotic and eukaryotic organisms such as E. coli, yeast, plants, insect cells, mammalian cells, including human cell lines and transgenic animals. Expression hosts can differ in their protein production levels as well as the types of post-translational modifications that are present on the expressed proteins. The choice of expression host can be made based on these and other factors, such as regulatory and safety considerations, production costs and the need and methods for purification.

Expression in eukaryotic hosts can include expression in yeasts such as Saccharomyces cerevisiae and Pichia pastoris, insect cells such as Drosophila cells and lepidopteran cells, plants and plant cells such as tobacco, corn, rice, algae, and lemna. Eukaryotic cells for expression also include mammalian cells lines such as Chinese hamster ovary (CHO) cells or baby hamster kidney (BHK) cells. Eukaryotic expression hosts also include production in transgenic animals, for example, including production in serum, milk and eggs. Transgenic animals for the production of wild-type Factor IX polypeptides are known in the art (U.S. Patent Publication Nos. 2002/0166130 and 2004/0133930) and can be adapted for production of modified Factor IX polypeptides provided herein.

Many expression vectors are available and known to those of skill in the art for the expression of Factor IX. The choice of expression vector is influenced by the choice of host expression system. Such selection is well within the level of skill of the skilled artisan. In general; expression vectors can include transcriptional promoters and optionally enhancers, translational signals, and transcriptional and translational termination signals. Expression vectors that are used for stable transformation typically have a selectable marker which allows selection and maintenance of the transformed cells. In some cases, an origin of replication can be used to amplify the copy number of the vectors in the cells.

Factor IX or modified Factor IX polypeptides also can be utilized or expressed as protein fusions. For example, a fusion can be generated to add additional functionality to a polypeptide. Examples of fusion proteins include, but are not limited to, fusions of a signal sequence, a tag such as for localization, e.g., a his6 tag or a myc tag, or a tag for purification, for example, a GST fusion, and a sequence for directing protein secretion and/or membrane association.

In one embodiment, the Factor IX polypeptide or modified Factor IX polypeptides can be expressed in an active form, whereby activation is achieved by incubation of the polypeptide activated Factor XI following secretion. In another embodiment, the protease is expressed in an inactive, zymogen form.

Methods of production of Factor IX polypeptides can include coexpression of one or more additional heterologous polypeptides that can aid in the generation of the Factor IX polypeptides. For example, such polypeptides can contribute to the post-translation processing of the Factor IX polypeptides. Exemplary polypeptides include, but are not limited to, peptidases that help cleave Factor IX precursor sequences, such as the propeptide sequence, and enzymes that participate in the modification of the Factor IX polypeptide, such as by glycosylation, hydroxylation, carboxylation, or phosphorylation, for example. An exemplary peptidase that can be coexpressed with Factor IX is PACE/furin (or PACE-SOL), which aids in the cleavage of the Factor IX propeptide sequence. An exemplary protein that aids in the carboxylation of the Factor IX polypeptide is the warfarin-sensitive enzyme vitamin K 2,3-epoxide reductase (VKOR), which produces reduced vitamin K for utilization as a cofactor by the vitamin K-dependent γ-carboxylase (Wajih et al., J. Biol. Chem. 280(36)31603-31607). A subunit of this enzyme, VKORC1, can be coexpressed with the modified Factor IX polypeptide to increase the γ-carboxylation. The one or more additional polypeptides can be expressed from the same expression vector as the Factor IX polypeptide or from a different vector.

a. Prokaryotic Expression

Prokaryotes, especially E. coli, provide a system for producing large amounts of Factor IX (see, for example, Platis et al. (2003) Protein Exp. Purif. 31(2): 222-30; and Khalilzadeh et al. (2004) J. Ind. Microbiol. Biotechnol. 31(2): 63-69). Transformation of E. coli is a simple and rapid technique well known to those of skill in the art. Expression vectors for E. coli can contain inducible promoters that are useful for inducing high levels of protein expression and for expressing proteins that exhibit some toxicity to the host cells. Examples of inducible promoters include the lac promoter, the trp promoter, the hybrid tac promoter, the T7 and SP6 RNA promoters and the temperature regulated λP_{L} promoter.

Factor IX can be expressed in the cytoplasmic environment of E. coli. The cytoplasm is a reducing environment and for some molecules, this can result in the formation of insoluble inclusion bodies. Reducing agents such as dithiothreitol and .beta.-mercaptoethanol and denaturants (e.g., such as guanidine-HCl and urea) can be used to resolubilize the proteins. An alternative approach is the expression of Factor IX in the periplasmic space of bacteria which provides an oxidizing environment and chaperonin-like and disulfide isomerases leading to the production of soluble protein. Typically, a leader sequence is fused to the protein to be expressed which directs the protein to the periplasm. The leader is then removed by signal peptidases inside the periplasm. Examples of periplasmic-targeting leader sequences include the pelB leader from the pectate lyase gene and the leader derived from the alkaline phosphatase gene. In some cases, periplasmic expression allows leakage of the expressed protein into the culture medium. The secretion of proteins allows quick and simple purification from the culture supernatant. Proteins that are not secreted can be obtained from the periplasm by osmotic lysis. Similar to cytoplasmic expression, in some cases proteins can become insoluble and denaturants and reducing agents can be used to facilitate solubilization and refolding. Temperature of induction and growth also can influence expression levels and solubility. Typically, temperatures between 25° C. and 37° C. are used. Mutations also can be used to increase solubility of expressed proteins. Typically, bacteria produce aglycosylated proteins. Thus, for the production of the hyperglycosylated Factor IX polypeptides provided herein, glycosylation can be added in vitro after purification from host cells.

b. Yeast

Yeasts such as Saccharomyces cerevisiae, Schizosaccharomyces pombe, Yarrowia lipolytica, Kluyveromyces lactis, and Pichia pastoris are useful expression hosts for FIX (see for example, Skoko et al. (2003) Biotechnol. Appl. Biochem. 38(Pt3):257-65). Yeast can be transformed with episomal replicating vectors or by stable chromosomal integration by homologous recombination. Typically, inducible promoters are used to regulate gene expression. Examples of such promoters include GAL1, GALT, and GALS and metallothionein promoters such as CUP1. Expression vectors often include a selectable marker such as LEU2, TRP1, HIS3, and URA3 for selection and maintenance of the transformed DNA. Proteins expressed in yeast are often soluble and co-expression with chaperonins, such as Bip and protein disulfide isomerase, can improve expression levels and solubility. Additionally, proteins expressed in yeast can be directed for secretion using secretion signal peptide fusions such as the yeast mating type alpha-factor secretion signal from Saccharomyces cerevisiae and fusions with yeast cell surface proteins such as the Aga2p mating adhesion receptor or the Arxula adeninivorans glucoamylase. A protease cleavage site (e.g., the Kex-2 protease) can be engineered to remove the fused sequences from the polypeptides as they exit the secretion pathway. Yeast also is capable of glycosylation at Asn-X-Ser/Thr motifs.

c. Insects and Insect Cells

Insects and insect cells, particularly using a baculovirus expression system, are useful for expressing polypeptides such as Factor IX or modified forms thereof (see, for example, Muneta et al. (2003) J. Vet. Med. Sci. 65(2):219-23). Insect cells and insect larvae, including expression in the haemolymph, express high levels of protein and are capable of most of the post-translational modifications used by higher eukaryotes. Baculoviruses have a restrictive host range which improves the safety and reduces regulatory concerns of eukaryotic expression. Typically, expression vectors use a promoter such as the polyhedrin promoter of baculovirus for high level expression. Commonly used baculovirus systems include baculoviruses such as Autographa californica nuclear polyhedrosis virus (AcNPV), and the Bombyx mori nuclear polyhedrosis virus (BmNPV) and an insect cell line such as Sf9 derived from Spodoptera frugzperda, Pseudaletia unipuncta (A7S) and Danaus plexippus (DpN1). For high level expression, the nucleotide sequence of the molecule to be expressed is fused immediately downstream of the polyhedrin initiation codon of the virus. Mammalian secretion signals are accurately processed in insect cells and can be used to secrete the expressed protein into the culture medium. In addition, the cell lines Pseudaletia unipuncta (A7S) and Danaus plexippus (DpN1) produce proteins with glycosylation patterns similar to mammalian cell systems.

An alternative expression system in insect cells is the use of stably transformed cells. Cell lines such as the Schnieder 2 (S2) and Kc cells (Drosophila melanogaster) and C7 cells (Aedes albopictus) can be used for expression. The Drosophila metallothionein promoter can be used to induce high levels of expression in the presence of heavy metal induction with cadmium or copper. Expression vectors are typically maintained by the use of selectable markers such as neomycin and hygromycin.

d. Mammalian Cells

Mammalian expression systems can be used to express Factor IX polypeptides. Expression constructs can be transferred to mammalian cells by viral infection such as adenovirus or by direct DNA transfer such as liposomes, calcium phosphate, DEAE-dextran and by physical means such as electroporation and microinjection. Expression vectors for mammalian cells typically include an mRNA cap site, a TATA box, a translational initiation sequence (Kozak consensus sequence) and polyadenylation elements. Such vectors often include transcriptional promoter-enhancers for high level expression, for example the SV40 promoter-enhancer, the human cytomegalovirus (CMV) promoter, and the long terminal repeat of Rous sarcoma virus (RSV). These promoter-enhancers are active in many cell types. Tissue and cell-type promoters and enhancer regions also can be used for expression. Exemplary promoter/enhancer regions include, but are not limited to, those from genes such as elastase I, insulin, immunoglobulin, mouse mammary tumor virus, albumin, alpha-fetoprotein, alpha 1-antitrypsin, beta-globin, myelin basic protein, myosin light chain-2, and gonadotropic releasing hormone gene control. Selectable markers can be used to select for and maintain cells with the expression construct. Examples of selectable marker genes include, but are not limited to, hygromycin B phosphotransferase, adenosine deaminase, xanthine-guanine phosphoribosyl transferase, aminoglycoside phosphotransferase, dihydrofolate reductase and thymidine kinase. Fusion with cell surface signaling molecules can direct expression of the proteins in an active state on the cell surface.

Many cell lines are available for mammalian expression including mouse, rat human, monkey, and chicken and hamster cells. Exemplary cell lines include, but are not limited to, BHK (i.e., BHK-21 cells), 293-F, CHO, CHO Express (CHOX; Excellgene), Balb/3T3, HeLa, MT2, mouse NS0 (non-secreting) and other myeloma cell lines, hybridoma and heterohybridoma cell lines, lymphocytes, fibroblasts, Sp2/0, COS, NIH3T3, HEK293, 293S, 293T, 2B8, and HKB cells. Cell lines also are available adapted to serum-free media which facilitates purification of secreted proteins from the cell culture media. One such example is the serum free EBNA-1 cell line (Pham et al., (2003) Biotechnol. Bioeng. 84:332-42). Expression of recombinant Factor IX polypeptides exhibiting similar structure and post-translational modifications as plasma-derived Factor IX are known in the art. Methods of optimizing vitamin K-dependent protein expression are known. For example, supplementation of vitamin K in culture medium or co-expression of vitamin K-dependent γ-carboxylases (Wajih et al., J. Biol. Chem. 280(36)31603-31607) can aid in post-translational modification of vitamin K-dependent proteins, such as Factor IX polypeptides.

e. Plants

Transgenic plant cells and plants can be used for the expression of Factor IX. Expression constructs are typically transferred to plants using direct DNA transfer such as microprojectile bombardment and PEG-mediated transfer into protoplasts, and with agrobacterium-mediated transformation. Expression vectors can include promoter and enhancer sequences, transcriptional termination elements, and translational control elements. Expression vectors and transformation techniques are usually divided between dicot hosts, such as Arabidopsis and tobacco, and monocot hosts, such as corn and rice. Examples of plant promoters used for expression include the cauliflower mosaic virus promoter, the nopaline synthase promoter, the ribose bisphosphate carboxylase promoter and the ubiquitin and UBQ3 promoters. Selectable markers such as hygromycin, phosphomannose isomerase and neomycin phosphotransferase are often used to facilitate selection and maintenance of transformed cells. Transformed plant cells can be maintained in culture as cells, aggregates (callus tissue) or regenerated into whole plants. Because plants have different glycosylation patterns than mammalian cells, this can influence the choice to produce Factor IX in these hosts. Transgenic plant cells also can include algae engineered to produce proteins (see, for example, Mayfield et al. (2003) Proc Natl Acad Sci USA 100:438-442). Because plants have different glycosylation patterns than mammalian cells, this can influence the choice to produce FIX in these hosts.

### IV. METHODS OF TREATING HEMOPHILIA AND BLEEDING DISORDERS

A. Therapeutic Regimens

The modified Factor IX polypeptides provided herein can be used for treatment of any condition for which unmodified Factor IX is employed. The modified polypeptides provided herein are designed to retain therapeutic activity but exhibit modified properties, particularly increased stability. Such modified properties, for example, can improve the therapeutic effectiveness of the polypeptides and/or can provide for additional routes of administration, such as oral administration. This section provides exemplary uses of and administration methods. These described therapies are exemplary and do not limit the applications of modified Factor IX polypeptides.

The modified Factor IX polypeptides provided herein can be used in various therapeutic as well as diagnostic methods in which Factor IX is employed. Such methods include, but are not limited to, methods of treatment of physiological and medical conditions described and listed below. Modified Factor IX polypeptides provided herein can exhibit improvement of in vivo activities and therapeutic effects compared to wild-type Factor IX, including lower dosage to achieve the same effect, a more sustained therapeutic effect and other improvements in administration and treatment.

The modified Factor IX polypeptides described herein exhibit increased protein stability and improved half-life. Thus, modified Factor IX polypeptides can be used to deliver longer-lasting, more stable therapies. Examples of therapeutic improvements using modified Factor IX polypeptides include, but are not limited to, lower dosages, fewer and/or less frequent administrations, decreased side effects and increased therapeutic effects.

In particular, modified Factor IX polypeptides, are intended for use in therapeutic methods in which Factor IX has been used for treatment. Such methods include, but are not limited to, methods of treatment of diseases and disorders, such as, but not limited to, blood coagulation disorders, hematologic diseases, hemorrhagic disorders, hemophilias, in particular hemophilia B, and acquired blood disorders, such as caused by liver disease. Modified Factor IX polypeptides also can be used in the treatment of additional bleeding diseases and disorders, such as, but not limited to, thrombocytopenia (such as, idiopathic thrombocytopenic purpura, and thrombotic thrombocytopenic purpura), Von Willebrand's disease, hereditary platelet disorders (such as, storage pool disease such as Chediak-Higashi and Hermansky-Pudlak syndromes, thromboxane A2 dysfunction, thromboasthenia, and Bernard-Soulier syndrome), hemolytic-uremic syndrome, Hereditary Hemorhhagic Telangiectsia, also known as Rendu-Osler-Weber syndrome, allergic purpura (Henoch Schonlein purpura) and disseminated intravascular coagulation. In some embodiments, the bleedings to be treated by Factor IX polypeptides occur in organs such as the brain, inner ear region, eyes, liver, lung, tumor tissue, gastrointestinal tract. In other embodiments, the bleeding is diffuse, such as in haemorrhagic gastritis and profuse uterine bleeding. Patients with bleeding disorders are often at risk for hemorrhage and excessive bleeding during surgery or trauma. Such patients often have acute haemarthroses (bleedings in joints), chronic haemophilic arthropathy, haematomas, (such as, muscular, retroperitoneal, sublingual and retropharyngeal), bleedings in other tissue, haematuria (bleeding from the renal tract), cerebral hemorrhage, surgery (such as, hepatectomy), dental extraction, and gastrointestinal bleedings (such as, UGI bleeds), that can be treated with modified Factor IX polypeptides. In one embodiment, the modified Factor IX polypeptides can be used to treat bleeding episodes due to trauma, or surgery, or lowered count or activity of platelets, in a subject. Exemplary methods for patients undergoing surgery include treatments to prevent hemorrhage and treatments before, during, or after surgeries such as, but not limited to, heart surgery, angioplasty, lung surgery, abdominal surgery, spinal surgery, brain surgery, vascular surgery, dental surgery, or organ transplant surgery, including transplantation of heart, lung, pancreas, or liver.

Factor IX polypeptides lacking functional peptidase activity have been used in therapeutic methods to inhibit blood coagulation (U.S. Pat. No. 6,315,995). Modified Factor IX polypeptides provided herein that inhibit or antagonize blood coagulation can be used in anticoagulant methods of treatment for ischemic disorders, such as a peripheral vascular disorder, a pulmonary embolus, a venous thrombosis, deep vein thrombosis (DVT), superficial thrombophlebitis (SVT), arterial thrombosis, a myocardial infarction, a transient ischemic attack, unstable angina, a reversible ischemic neurological deficit, an adjunct thrombolytic activity, excessive clotting conditions, reperfusion injury, sickle cell anemia or stroke disorder. In patients with an increased risk of excessive clotting, such as DVT or SVT, during surgery, protease inactive modified Factor IX polypeptides provided herein can be administered to prevent excessive clotting in surgeries, such as, but not limited to heart surgery, angioplasty, lung surgery, abdominal surgery, spinal surgery, brain surgery, vascular surgery, or organ transplant surgery, including transplantation of heart, lung, pancreas, or liver. In some cases treatment is performed with Factor IX alone. In some cases, Factor IX is administered in conjunction with additional coagulation or anticoagulation factors as required by the condition or disease to be treated.

Treatment of diseases and conditions with modified Factor IX polypeptides can be effected by any suitable route of administration using suitable formulations as described herein including, but not limited to, injection, pulmonary, oral and transdermal administration. If necessary, a particular dosage and duration and treatment protocol can be empirically determined or extrapolated. For example, exemplary doses of recombinant and native Factor IX polypeptides can be used as a starting point to determine appropriate dosages. For example, a recombinant Factor IX polypeptide, BeneFIX®, has been administered to patients with hemophilia B, at a dosage of 50 I.U./kg over a 10 minute infusion, resulting in a mean circulating activity of 0.8.+/−.0.2 I.U./dL per I.U./kg infused with a mean half-life of 19.4.+/−.5.4 hours. Modified Factor IX polypeptides that are more stable and have an increased half-life in vivo can be effective at reduced dosage amounts and or frequencies. For example, because of the improvement in properties such as serum stability, dosages can be lower than comparable amounts of unmodified Factor IX. Dosages for unmodified Factor IX polypeptides can be used as guidance for determining dosages for modified Factor IX polypeptides. Factors such as the level of activity and half-life of the modified Factor IX in comparison to the unmodified Factor IX can be used in making such determinations. Particular dosages and regimens can be empirically determined.

Dosage levels and regimens can be determined based upon known dosages and regimens, and, if necessary can be extrapolated based upon the changes in properties of the modified polypeptides and/or can be determined empirically based on a variety of factors. Such factors include body weight of the individual, general health, age, the activity of the specific compound employed, sex, diet, time of administration, rate of excretion, drug combination, the severity and course of the disease, and the patient's disposition to the disease and the judgment of the treating physician. The active ingredient, the polypeptide, typically is combined with a pharmaceutically effective carrier. The amount of active ingredient that can be combined with the carrier materials to produce a single dosage form or multi-dosage form can vary depending upon the host treated and the particular mode of administration.

The effect of the Factor IX polypeptides on the clotting time of blood can be monitored using any of the clotting tests known in the art including, but not limited to, whole blood partial thromboplastin time (PTT), the activated partial thromboplastin time (aPTT), the activated clotting time (ACT), the recalcified activated clotting time, or the Lee-White Clotting time.

Upon improvement of a patient's condition, a maintenance dose of a compound or compositions can be administered, if necessary; and the dosage, the dosage form, or frequency of administration, or a combination thereof can be modified. In some cases, a subject can require intermittent treatment on a long-term basis upon any recurrence of disease symptoms or based upon scheduled dosages. In other cases, additional administrations can be required in response to acute events such as hemorrhage, trauma, or surgical procedures.

B. Combined Therapy

In another embodiment, it is envisioned to use an inhibitor as described herein combination with other therapeutic modalities. Thus, in addition to the Factor IX variant therapies described herein, one may also provide to the patient more “standard” pharmaceutical therapies for hemophilia B. Examples of other therapies include, without limitation, non-mutated Factor IX therapy, or other additional coagulation factors selected from plasma purified or recombinant coagulation factors, procoagulants, such as vitamin K, vitamin K derivative and protein C inhibitors, plasma, platelets, red blood cells and corticosteroids.

Combinations may be achieved by administering a single composition or pharmacological formulation that includes both agents, or by administering two distinct compositions or formulations at the same time, wherein one composition includes the Factor IX variants of the present disclosure, and the other includes the “standard” agent. Alternatively, the therapy using the Factor IX variants of the present disclosure may precede or follow administration of the other agent by intervals ranging from minutes to weeks. In embodiments where the other agent and the Factor IX variants are administered separately, one would generally ensure that a significant period of time did not expire between each delivery, such that the agent and Factor IX variant would still be able to exert an advantageously combined effect on the subject. In such instances, it is contemplated that one would typically contact the cell with both modalities within about 12-24 hours of each other and, more preferably, within about 6-12 hours of each other, with a delay time of only about 12 hours being most preferred. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

It also is contemplated that patients will receive more than one administration of the Factor IX variant according to the present disclosure, and/or the other agent. In this regard, various combinations may be employed. By way of illustration, where the Factor IX variant of according to the present disclosure is “A” and the other agent is “B”, the following permutations based on 3 and 4 total administrations are exemplary:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are likewise contemplated.

C. Pharmacological Formulations and Routes for Administration

Pharmacological therapeutic agents and methods of administration, dosages, etc., are well known to those of skill in the art (see for example, the “Physicians Desk Reference,” Klaassen's “The Pharmacological Basis of Therapeutics,” “Remington's Pharmaceutical Sciences,” and “The Merck Index, Eleventh Edition,” incorporated herein by reference in relevant parts), and may be combined with the disclosure in light of the disclosures herein. Some variation in dosage will necessarily occur depending on the condition of the subject being treated. The person responsible for administration will, in any event, determine the appropriate dose for the individual subject, and such individual determinations are within the skill of those of ordinary skill in the art.

Where clinical applications are contemplated, pharmaceutical compositions will be prepared in a form appropriate for the intended application. Generally, this will entail preparing compositions that are essentially free of pyrogens, as well as other impurities that could be harmful to humans or animals.

One will generally desire to employ appropriate salts and buffers to render delivery vectors stable and allow for uptake by target cells. Buffers also will be employed when recombinant cells are introduced into a patient. Aqueous compositions of the present disclosure comprise an effective amount of the vector or cells, dissolved or dispersed in a pharmaceutically acceptable carrier or aqueous medium. The phrase “pharmaceutically or pharmacologically acceptable” refers to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Except insofar as any conventional media or agent is incompatible with the active ingredients of the present disclosure, its use in therapeutic compositions is contemplated. Supplementary active ingredients also can be incorporated into the compositions, provided they do not inactivate the vectors or cells of the compositions.

The active compositions of the present disclosure may include classic pharmaceutical preparations. Administration of these compositions according to the present disclosure may be via any common route so long as the target tissue is available via that route. This includes oral, nasal, or buccal. Alternatively, administration may be by intradermal, subcutaneous, intramuscular, intraperitoneal or intravenous injection, or by direct injection into cardiac tissue. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

The polypeptides can be formulated as the sole pharmaceutically active ingredient in the composition or can be combined with other active ingredients. The polypeptides can be targeted for delivery, such as by conjugation to a targeting agent, such as an antibody. Liposomal suspensions, including tissue-targeted liposomes, also can be suitable as pharmaceutically acceptable carriers. These can be prepared according to methods known to those skilled in the art. For example, liposome formulations can be prepared as described in U.S. Pat. No. 4,522,811. Liposomal delivery also can include slow release formulations, including pharmaceutical matrices such as collagen gels and liposomes modified with fibronectin (see, for example, Weiner et al. (1985) J Pharm Sci. 74(9): 922-5).

The active compound is included in the pharmaceutically acceptable carrier in an amount sufficient to exert a therapeutically useful effect in the absence of undesirable side effects on the subject treated. The therapeutically effective concentration can be determined empirically by testing the compounds in known in vitro and in vivo systems, such as the assays provided herein. The active compounds can be administered by any appropriate route, for example, oral, nasal, pulmonary, parenteral, intravenous, intradermal, subcutaneous, or topical, in liquid, semi-liquid or solid form and are formulated in a manner suitable for each route of administration. In a particular embodiment, the Factor IX polypeptide is administered orally. Factor IX polypeptides can be formulated with additional coagulation factors.

The modified Factor IX and physiologically acceptable salts and solvates can be formulated for administration by inhalation (either through the mouth or the nose), oral, transdermal, pulmonary, parenteral, or rectal administration or injection. For administration by inhalation, the modified Factor IX can be delivered in the form of an aerosol spray presentation from pressurized packs or a nebulizer with the use of a suitable propellant, such as, dichlorodifluoromethane, trichlorofluoromethane, dichlorotetrafluoroethane, carbon dioxide or other suitable gas. In the case of a pressurized aerosol, the dosage unit can be determined by providing a valve to deliver a metered amount. Capsules and cartridges of such as, gelatin for use in an inhaler or insufflator can be formulated containing a powder mix of a therapeutic compound and a suitable powder base such as lactose or starch.

Factor IX polypeptides can be formulated as liquid or powder. In the case of a liquid, the modified polypeptides can be injected from a syringe or an auto-injector. In the case of a powder, the modified polypeptides can be reconstituted with a pharmaceutically acceptable excipient, such as pharmaceutically-acceptable saline, prior to administration. Administration can be by a medical professional or self-administration.

For pulmonary administration to the lungs, the modified Factor IX can be delivered in the form of an aerosol spray presentation from a nebulizer, turbonebulizer, or microprocessor-controlled metered dose oral inhaler with the use of a suitable propellant. Generally, the particle size is small, such as in the range of 0.5 to 5 microns. In the case of a pharmaceutical composition formulated for pulmonary administration, detergent surfactants are not typically used. Pulmonary drug delivery is a promising non-invasive method of systemic administration. The lungs represent an attractive route for drug delivery, mainly due to the high surface area for absorption, thin alveolar epithelium, extensive vascularization, lack of hepatic first-pass metabolism, and relatively low metabolic activity.

The modified Factor IX polypeptides exhibit increased resistance to proteolysis and half-life in the gastrointestinal tract. Thus, preparations for oral administration can be suitably formulated without the use of protease inhibitors, such as a Bowman-Birk inhibitor, a conjugated Bowman-Birk inhibitor, aprotinin and camostat.

The modified Factor IX polypeptides can be formulated as a depot preparation. Such long-acting formulations can be administered by implantation (for example, subcutaneously or intramuscularly) or by intramuscular injection. Thus, for example, the therapeutic compounds can be formulated with suitable polymeric or hydrophobic materials (for example as an emulsion in an acceptable oil) or ion exchange resins, or as sparingly soluble derivatives, for example, as a sparingly soluble salt.

The modified Factor IX can be formulated, for example, for parenteral administration by injection (such as, by bolus injection or continuous infusion). Formulations for injection can be presented in unit dosage form (such as, in ampoules or in multi-dose containers) with an added preservative. The compositions can take such forms as suspensions, solutions or emulsions in oily or aqueous vehicles and can contain formulatory agents such as suspending, stabilizing and/or dispersing agents. Alternatively, the active ingredient can be in powder-lyophilized form for constitution with a suitable vehicle, such as, sterile pyrogen-free water, before use.

The pharmaceutical compositions can be formulated for local or topical application, such as for topical application to the skin (transdermal) and mucous membranes, such as in the eye, in the form of gels, creams, and lotions and for application to the eye or for intracisternal or intraspinal application. Such solutions, particularly those intended for ophthalmic use, can be formulated as 0.01%-10% isotonic solutions and pH about 5-7 with appropriate salts. The compounds can be formulated as aerosols for topical application, such as by inhalation (see, for example, U.S. Pat. Nos. 4,044,126, 4,414,209 and 4,364,923, which describe aerosols for delivery of a steroid useful for treatment inflammatory diseases, particularly asthma).

The concentration of active compound in the drug composition depends on absorption, inactivation and excretion rates of the active compound, the dosage schedule, and amount administered as well as other factors known to those of skill in the art. As described further herein, dosages can be determined empirically using dosages known in the art for administration of unmodified Factor IX, and comparisons of properties and activities (such as, stability and activities) of the modified Factor IX compared to the unmodified and/or native Factor IX.

For oral administration, the pharmaceutical compositions can take the form of, for example, tablets, pills, liquid suspensions, or capsules prepared by conventional means with pharmaceutically acceptable excipients such as binding agents (such as, pregelatinized maize starch, polyvinylpyrrolidone or hydroxypropyl methylcellulose); fillers (such as, lactose, microcrystalline cellulose or calcium hydrogen phosphate); lubricants (such as, magnesium stearate, talc or silica); disintegrants (such as, potato starch or sodium starch glycolate); or wetting agents (such as, sodium lauryl sulphate). The tablets can be coated by methods well known in the art. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically-acceptable saline, pharmaceutically acceptable additives such as suspending agents (such as, sorbitol syrup, cellulose derivatives or hydrogenated edible fats); emulsifying agents (such as, lecithin or acacia); non-aqueous vehicles (such as, almond oil, oily esters, ethyl alcohol or fractionated vegetable oils); and preservatives (such as, methyl or propyl-p-hydroxybenzoates or sorbic acid). The preparations also can contain buffer salts, flavoring, coloring and sweetening agents as appropriate.

The modified Factor IX polypeptides can be formulated for oral administration, Oral formulations include tablets, capsules, liquids or other suitable vehicle for oral administration. In some examples, the capsules or tablets are formulated with an enteric coating to be gastro-resistant. Preparation of pharmaceutical compositions containing a modified Factor IX for oral delivery can include formulating modified Factor IX polypeptides with oral formulations known in the art and/or those described herein. The compositions as formulated do not require addition of protease inhibitors and/or other ingredients that are necessary for stabilization of unmodified (for protease resistance) and wild-type Factor IX polypeptides upon exposure to proteases, such as selecting pH and other conditions to minimize protease cleavage. For example, such compositions exhibit stability in the absence of compounds such as actinonin or epiactinonin and derivatives thereof; Bowman-Birk inhibitor and conjugates thereof; aprotinin and camostat. In other examples, the preparations for oral administration can include protease inhibitors.

For oral administration, the pharmaceutical compositions can be prepared by conventional means with pharmaceutically acceptable excipients such as binding agents (such as, pre-gelatinized maize starch, polyvinylpyrrolidone or hydroxypropyl methylcellulose); fillers (such as, lactose, microcrystalline cellulose or calcium hydrogen phosphate); lubricants (such as, magnesium stearate, talc or silica); disintegrants (such as, potato starch or sodium starch glycolate); or wetting agents (such as, sodium lauryl sulphate). The active ingredient present in the capsule can be in, for example, liquid or lyophilized form. The tablets or capsules can be coated by methods well known in the art. Tablets and capsules can be coated, for example, with an enteric coating. Liquid preparations for oral administration can take the form of, for example, solutions, syrups or suspensions, or they can be presented as a dry product for constitution with water or other suitable vehicle before use. Such liquid preparations can be prepared by conventional means with pharmaceutically acceptable additives such as suspending agents (such as, sorbitol syrup, cellulose derivatives or hydrogenated edible fats); emulsifying agents (such as, lecithin or acacia); non aqueous vehicles (such as, almond oil, oily esters, ethyl alcohol or fractionated vegetable oils); and preservatives (such as, methyl or propyl-p hydroxybenzoates or sorbic acid). The preparations also can contain buffer salts, flavoring, coloring and/or sweetening agents as appropriate.

Preparations for oral administration can be formulated to give controlled or sustained release or for release after passage through the stomach or in the small intestine of the active compound. For oral administration the compositions can take the form of tablets, capsules, liquids, lozenges and other forms suitable for oral administration. Formulations suitable for oral administration include lozenges and other formulations that deliver the pharmaceutical composition to the mucosa of the mouth, throat and/or gastrointestinal tract. Lozenges can be formulated with suitable ingredients including excipients for example, anhydrous crystalline maltose and magnesium stearate. As noted, modified polypeptides described herein exhibit resistance to blood or intestinal proteases.

The compositions for oral administration can be formulated, for example, as gastro-resistant capsules or tablets. Such gastro-resistant capsules are modified release capsules that are intended to resist the gastric fluid and to release their active ingredient or ingredients in the intestinal fluid. They are prepared by providing hard or soft capsules with a gastro-resistant shell (enteric capsules) or by filling capsules with granules or with particles covered with a gastro-resistant coating.

The enteric coating is typically, although not necessarily, a polymeric material. Enteric coating materials can contain bioerodible, gradually hydrolyzable and/or gradually water-soluble polymers. The “coating weight,” or relative amount of coating material per capsule, generally dictates the time interval between ingestion and drug release. Any coating should be applied to a sufficient thickness such that the entire coating does not dissolve in the gastrointestinal fluids at pH below about 5, but does dissolve at pH about 5 and above. It is expected that any anionic polymer exhibiting a pH-dependent solubility profile can be used as an enteric coating to achieve delivery of the active ingredient to the lower gastrointestinal tract. The selection of the specific enteric coating material will depend on the following properties: resistance to dissolution and disintegration in the stomach; impermeability to gastric fluids and drug/carrier/enzyme while in the stomach; ability to dissolve or disintegrate rapidly at the target intestine site; physical and chemical stability during storage; non-toxicity; ease of application as a coating (substrate friendly); and economical practicality.

Suitable enteric coating materials include, but are not limited to: cellulosic polymers, such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, hydroxypropylmethyl cellulose succinate and carboxymethylcellulose sodium; acrylic acid polymers and copolymers, such as formed from acrylic acid, met acrylic acid, methyl acrylate, ammonium methylacrylate, ethyl acrylate, methyl methacrylate and/or ethyl methacrylate (such as, those copolymers sold under the trade name EUDRAGIT); vinyl polymers and copolymers, such as polyvinyl pyrrolidone (PVP), polyvinyl acetate, polyvinyl acetate phthalate, vinyl acetate crotonic acid copolymer, and ethylene-vinyl acetate copolymers; and shellac (purified lac). Combinations of different coating materials also can be used to coat a single capsule. Exemplary of such gastro-resistant capsules are hard gelatin capsules (sold by Torpac or Capsugel) size 9, coated with cellulose acetate phthalate (CAP) at 12% in acetone.

The enteric coating provides for controlled release of the active agent, such that drug release can be accomplished at some generally predictable location in the lower intestinal tract below the point at which drug release would occur without the enteric coating. The enteric coating also prevents exposure of the hydrophilic therapeutic agent and carrier to the epithelial and mucosal tissue of the buccal cavity, pharynx, esophagus, and stomach, and to the enzymes associated with these tissues. The enteric coating therefore helps to protect the active agent and a patient's internal tissue from any adverse event prior to drug release at the desired site of delivery. Furthermore, the coated capsules can permit optimization of drug absorption, active agent protection, and safety. Multiple enteric coatings targeted to release the active agent at various regions in the lower gastrointestinal tract would enable even more effective and sustained improved delivery throughout the lower gastrointestinal tract.

The coating optionally can contain a plasticizer to prevent the formation of pores and cracks that would permit the penetration of the gastric fluids. Suitable plasticizers include, but are not limited to, triethyl citrate (CITROFLEX 2), triacetin (glyceryl triacetate), acetyl triethyl citrate (CITROFLEC A2), CARBOWAX 400 (polyethylene glycol 400), diethyl phthalate, tributyl citrate, acetylated monoglycerides, glycerol, fatty acid esters, propylene glycol, and dibutyl phthalate. In particular, a coating comprised of an anionic carboxylic acrylic polymer will typically contain less than about 50% by weight, such as less than about 30%, 10% to about 25% by weight, based on the total weight of the coating, of a plasticizer, particularly dibutyl phthalate, polyethylene glycol, triethyl citrate and triacetin. The coating also can contain other coating excipients, such as detackifiers, antifoaming agents, lubricants (such as, magnesium stearate), and stabilizers (such as, hydroxypropylcellulose, acids and bases) to solubilize or disperse the coating material, and to improve coating performance and the coated product.

The coating can be applied to the capsule or tablet using conventional coating methods and equipment. For example, an enteric coating can be applied to a capsule using a coating pan, an airless spray technique, fluidized bed coating equipment, or the like. Detailed information concerning materials, equipment and processes for preparing coated dosage forms are described in Pharmaceutical Dosage Forms: Tablets, eds. Lieberman et al. (New York: Marcel Dekker, Inc., 1989), and in Ansel et al., Pharmaceutical Dosage Forms and Drug Delivery Systems, 6.sup.th Edition (Media, Pa.: Williams & Wilkins, 1995). The coating thickness, as noted above, must be sufficient to ensure that the oral dosage form remains intact until the desired site of topical delivery in the lower intestinal tract is reached.

Preparations for oral administration can be formulated to give controlled or sustained release or for release after passage through the stomach or in the small intestine of the active compound. For oral administration the compositions can take the form of tablets, capsules, liquids, lozenges and other forms suitable for oral administration Formulations suitable for oral administration include lozenges and other formulations that deliver the pharmaceutical composition to the mucosa of the mouth, throat and/or gastrointestinal tract. Lozenges can be formulated with suitable ingredients including excipients for example, anhydrous crystalline maltose and magnesium stearate. As noted, modified polypeptides herein exhibit resistance to blood or intestinal proteases and can exhibit increased half-life in the gastrointestinal tract. Thus, preparations of oral administration can be suitably formulated without additional protease inhibitors or other protective compounds, such as a Bowman-Birk inhibitor, a conjugated Bowman-Birk inhibitor, aprotinin and camostat. Preparations for oral administration also can include a modified FIX resistant to proteolysis formulated with one or more additional ingredients that also confer proteases resistance, or confer stability in other conditions, such as particular pH conditions.

The compositions of the present disclosure generally may be formulated in a neutral or salt form. Pharmaceutically-acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety, and purity standards as required by FDA Office of Biologics standards.

### V. PURIFICATION OF PROTEINS

It will be desirable to purify peptides and polypeptides according to the present disclosure. Protein purification techniques are well known to those of skill in the art. These techniques involve, at one level, the crude fractionation of the cellular milieu to polypeptide and non-polypeptide fractions. Having separated the polypeptide from other proteins, the polypeptide of interest may be further purified using chromatographic and electrophoretic techniques to achieve partial or complete purification (or purification to homogeneity). Analytical methods particularly suited to the preparation of a pure peptide are ion-exchange chromatography, exclusion chromatography; polyacrylamide gel electrophoresis; isoelectric focusing. A particularly efficient method of purifying peptides is fast protein liquid chromatography or even HPLC.

Certain aspects of the present disclosure concern the purification, and in particular embodiments the substantial purification, of an encoded protein or peptide. The term “purified protein or peptide” as used herein, is intended to refer to a composition, isolatable from other components, wherein the protein or peptide is purified to any degree relative to its naturally-obtainable state. A purified protein or peptide therefore also refers to a protein or peptide, free from the environment in which it may naturally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the proteins in the composition.

Various methods for quantifying the degree of purification of the protein or peptide will be known to those of skill in the art in light of the present disclosure. These include, for example, determining the specific activity of an active fraction, or assessing the amount of polypeptides within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the expressed protein or peptide exhibits a detectable activity.

Various techniques suitable for use in protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified protein or peptide.

There is no general requirement that the protein or peptide always be provided in their most purified state. Indeed, it is contemplated that less substantially purified products will have utility in certain embodiments. Partial purification may be accomplished by using fewer purification steps in combination, or by utilizing different forms of the same general purification scheme. For example, it is appreciated that a cation-exchange column chromatography performed utilizing an HPLC apparatus will generally result in a greater “-fold” purification than the same technique utilizing a low pressure chromatography system. Methods exhibiting a lower degree of relative purification may have advantages in total recovery of protein product, or in maintaining the activity of an expressed protein.

It is known that the migration of a polypeptide can vary, sometimes significantly, with different conditions of SDS/PAGE (Capaldi et al., 1977). It will therefore be appreciated that under differing electrophoresis conditions, the apparent molecular weights of purified or partially purified expression products may vary.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present disclosure is discussed below.

### VI. ASSAYS FOR FACTOR IX ACTIVITY

In another aspect of the disclosure, the inventors have developed an assay to detect Factor IX activity in plasma. Factor IXa (FIXa) is unique among the coagulation serine proteases for 3 major reasons. First, Factor IXa is a principal product of tissue factor-factor VIIa (TF-FVIIa) complex in the presence of physiological inhibitors. Second, Factor Xa generation by the Factor IXa-Factor Villa complex is rate-limiting for plasma thrombin generation. And third, isolated Factor IXa is poorly reactive with both substrate and inhibitors. Modeling of blood coagulation suggests that subnanomolar concentrations of Factor IXa are sufficient to support plasma thrombin generation; however, most assays require greater than nanomolar FIXa concentrations for detection. Thus, the current ability to measure circulating levels of Factor IXa is limited at best, and necessary to accurately portray the biologic roles of this enzyme. The inventors' laboratory has developed a novel, highly sensitive assay to detect physiologically relevant levels of Factor IXa activity in patient plasma (or other body fluids) based on a modified thrombin generation assay in FIX-deficient plasma.

The enhanced thrombin generation assay, or “ETGA,” detects Factor IXa activity in test samples by dilution into citrated FIX-deficient plasma system. Briefly, a standard curve is established by adding 10 μl of test plasma containing 0-80 pM human Factor IXa to 50 μl of Factor IX-deficient plasma. Simultaneously, human Factor VIII (19.2 nM) was activated with 12.8 nM thrombin for 30 sec, neutralized with 1.25-fold molar excess of hirudin, and the resulting thrombin-activated Factor Villa was added to plasma (final plasma concentration 1.3 nM) immediately after recalcification with the fluorogenic substrate. Plasma thrombin generation (TG) was detected by cleavage of fluorogenic substrate Z-Gly-Gly-Arg-AMC in a Biotek Synergy HT plate reader, and fluorescent data exported to TECHNOTHROMBIN TGA Evaluation Software. Software generated TG parameters including lag time, peak thrombin concentration, time to thrombin peak and velocity index. Factor IXa concentration was plotted versus mean peak thrombin±SEM (n=3) and the data fit to a parabolic function. Sample Factor IXa activity was obtained from the standard curve using mean peak thrombin concentration. The specificity of the TG response is determined by pre-incubation of test plasma with inhibitory antibodies. To block activity due to contact pathway-dependent Factor IXa generation during the assay, activity is determined in the presence of the monoclonal antibody 01A6 which blocks Factor IX activation by Factor XIa. Similarly, the Factor IXa dependence of the activity is verified by pre-incubation test plasma with an inhibitory anti-Factor IX Gla domain antibody. An inhibitory anti-TF antibody had no effect on plasma activity in this assay.

The assay was initially employed to evaluate circulating Factor IXa activity in plasma obtained from ovarian cancer patients. Additionally, the assay has been used to detect trace Factor IXa contamination of recombinant Factor IX zymogen preparations, and determine the plasma half-life of Factor IXa in human plasma. It should also be useful for detecting trace Factor IXa concentrations in other plasma-derived protein preparations or aging plasma products.

Westmark et al., J. Thrombosis Haemostasis, 13:1053-1063 (2015), describing such an assay, is hereby incorporated by reference.

### VII. EXAMPLES

The following examples are included to further illustrate various aspects of the disclosure. It should be appreciated by those of skill in the art that the techniques disclosed in the examples which follow represent techniques and/or compositions discovered by the inventors to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Materials and Methods

Materials.

Normal, pooled and FIX-deficient human plasmas were purchased from HRF (Raleigh, N.C.) and Diagnostica Stago (Toronto, Ontario-Canada) and thrombin calibrator (thrombin-α2-macroglobulin complex) from Diagnostica Stago; corn trypsin inhibitor (CTI) from Haematologic Technologies (Essex Junction, Vt.); human plasma-derived antithrombin, factors IX, IXa and XIa, and primary antibodies from EnzymeResearch Laboratories (South Bend, Ind.); phosphatidylserine (PS) and phosphatidylcholine (PC) from Avanti Polar Lipids (Alabaster, Ala.); cholesterol (C) from Calbiochem (San Diego, Calif.); PC:PS:C (molar ratio 75:25:1) phospholipid vesicles were prepared by extrusion through a 100-nm polycarbonate filter (MacDonald et al., 1991); porcine intestinal UFH and bovine serum albumin (BSA) (A-9647) from Sigma-Aldrich (St Louis, Mo.); Vitamin K1 from Hospira, Inc. (Lake Forest, Ill.); restriction enzymes from New England Biolabs (Ipswich, Mass.); QuikChange site-directed mutagenesis kit from Agilent Technologies (Cedar Creek, Tex.); FuGENE 6 from Promega (Madison, Wis.); Z-Gly-Gly-Arg-AMC.HCl from Bachem Biosciences (King of Prussia, Pa.); and Pefafluor FIXa® from DSM Nutritional Product LTD (Aesch, Switzerland).

DNA Mutagenesis and Plasmid Constructions.

Mammalian expression vector pcDNA3.1 (Life Technologies, Grand Island, N.Y.) was used for cloning and expression. Factor IX wild-type (WT) was constructed as described previously (Krishnaswamy, S., 2005; Misenheimer et al., 2007). Site-directed mutagenesis was performed using QuikChange II XL kit following manufacturer's instructions. Mutagenesis primers are listed in Table 4.

Expression and Purification of rFIX.

A HEK293 cell line stably transfected with vitamin K epoxide reductase (VKOR/HEK293) was provided by Darrel Stafford (University of North Carolina-Chapel Hill). Stable VKOR/HEK293 cell lines expressing human Factor IX WT, K126A, K132A, K126A/K132A, R150A, K126A/R150A, K132A/R150A and K126A/K132A/R150A were constructed via transfection with mutant pcDNA3.1-Factor IX plasmids using FuGENE 6 lipid. Recombinatn Factor IX proteins were purified to homogeneity from conditioned media and quantitated (Yuan et al., 2005). Recombinant Factor IX was activated to recombinant Factor IXa with human Factor XIa and catalytic sites quantitated by titration with antithrombin (Misenheimer et al., 2007).

SDS-PAGE Analysis of Purified Recombinant Proteins.

Fifty ng (western blot) or 1 μg (Coomassie) protein was run on a 10% Next Gel (Amresco, Solon, Ohio) and stained with GelCode Blue Safe Stain (ThermoSci, Waltham, Mass.) or western blotted with polyclonal donkey anti-human FIX-HRP conjugated antibody (1:10,000 dilution) and Pierce® ECL 2 HRP Western Blot Substrate (Pierce Chemical, Rockford, Ill.) via film.

Plasma Coagulant Activity.

Coagulant activity was determined in an activated partial thromboplastin time (APTT) assay by addition of recombinant FIX(a) to Factor IX-deficient plasma just prior to recalcification (Misenheimer et al., 2007). Clotting times were determined using a STArt 4 coagulometer, and activity of the proteins was determined by comparison to a standard curve constructed by diluting normal pooled plasma into FIX-deficient plasma.

Plasma Thrombin Generation Assay (TGA).

Plasma thrombin generation was detected by cleavage of fluorogenic substrate Z-Gly-Gly-Arg-AMC in a Biotek Synergy HT plate reader equipped with GEN 5 software (Biotek Instruments, Winooski, Vt., USA) [30]. A calibration curve was constructed with thrombin calibrator at final plasma concentrations of 5-500 nM. The ability of FIX to support tissue factor (TF)-triggered thrombin generation was assessed by adding initiator solution as described in (Buyue et al., 2008) to 60 Vl of FIX-deficient plasma, with preheating at 37° C. for 10 min. Assays were then initiated with fluorogenic substrate and calcium solution (FluCal))Buyue et al., 2008). Final concentrations (extrapolated to 60 Vl plasma) were 0.2 pM TF, 8.3 VM PC:PS:C vesicles, 40 Vg/mL CTI and 0-90 nM plasma-derived FIX (pFIX) or rFIX. FIXa-triggered thrombin generation was similarly assessed, except TF was omitted from the initiator solution, and FIX was replaced with FIXa (0-100 pM final plasma concentrations). Fluorescent signal data was exported to TECHNOTHROMBIN TGA Evaluation Software (Technoclone GmbHVienna, Austria), and thrombin generation over time was determined using the calibration curve. Software generated parameters to describe each thrombin generation curve included: lag time, peak thrombin concentration, time to thrombin peak and velocity index (slope between end of lag time and peak thrombin).

Enhanced Thrombin Generation Assay (ETGA).

To detect physiologically relevant FIXa concentrations, the FIXa-triggered TGA was modified as follows: sample plasma (15 μl) containing FIXa was added to FIX-deficient plasma (45 μl) and warmed to 37° C. Simultaneously, human FVIII (19.2 nM) was activated with 12.8 nM thrombin for 30 sec, neutralized with 1.25-fold molar excess of hirudin, and the resulting thrombin-activated FVIIIa was added to plasma (final plasma concentration 1.3 nM) immediately after recalcification with FluCal. The TGA in the absence of TF was performed as described. A standard curve generated using human pFIXa (0-80 pM) (n=3) was plotted versus peak thrombin concentration and fit to a parabolic function. FIXa concentration in the plasma sample is obtained from the standard curve using mean peak thrombin concentration.

Inhibition of FIXa by Antithrombin.

The rate of inhibition by antithrombin was determined under pseudo-first order conditions in a discontinuous assay sampled overtime to determine residual protease activity. A 275 VL reaction containing 292.5 nM rFIXa and 4.5 μM antithrombin in tenase buffer (0.15 M NaCl, 20 mM HEPES, pH 7.4, 2 mM CaCl₂, 1 mg/mL BSA, 0.1% PEG-8000) at RT was sampled over 120 min. Identical reactions, in the presence of synthetic pentasaccharide Fondaparinux (460 nM) or unfractionated heparin (UFH, 0.24 U/ml), were sampled over 2-40 min. Reaction aliquots (22 μL) were added to 78 μL of a fluorogenic substrate reaction to yield final concentrations of 250 VM Pefafluor FIXa® (CH3SO2-D-CHG-Gly-Arg-AMC.AcOH), 30% ethylene glycol and 1 mg/mL Polybrene® in tenase buffer (n=3-5, ±SEM). Initial rates of substrate hydrolysis (μI) were determined from the slope of the fluorescence intensity change (360/40 nm-excitation, 460/40 nm-emission) over 10 min at RT. The rate of hydrolysis was converted to residual enzyme concentration using a standard curve constructed with pFIXa protein (0-80 nM). Inhibition rate constants were determined by fitting the data to the equation Et=(E0)e−k′t to obtain the apparent rate constant (k′), where E0 is the initial enzyme activity and Et is the enzyme activity at time t. The rate constant k′ is divided by antithrombin concentration (4.5 μM) to obtain the estimated second order rate constant (k2). Inhibition rate constants were expressed as mean value±standard error of the mean (SEM) (n=3-4).

Determination of the Half-Life of FIXa Activity in Human Plasma.

rFIXa (50-400 pM) was incubated in FIX-deficient plasma for 0-120 min at 37° C. prior to sampling into the ETGA assay. The resulting concentrations were plotted versus time and fit to the first order decay equation A=A0e−kt, where A is activity, t is time, and k is rate constant. Protease half-life (t1/2) was obtained from 0.693/k and expressed as mean t1/2±SEM (n=3).

Statistical Analysis.

Graphs, tables and associated statistics were generated using Microsoft Excel™ 2010 (version 14) (Microsoft Corporation, Redmond, Wash.) or KaleideGraph (version 4.5.) (Synergy Software, Reading, Pa.). Mean values±SEM were calculated (n>3) and data were compared for significant differences using the unpaired Student t-test.

### Example 2—Results

Expression, Purification and Activation of Human rFIX.

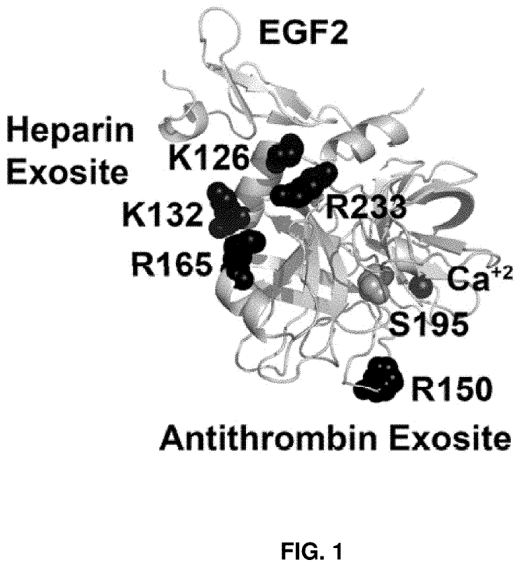

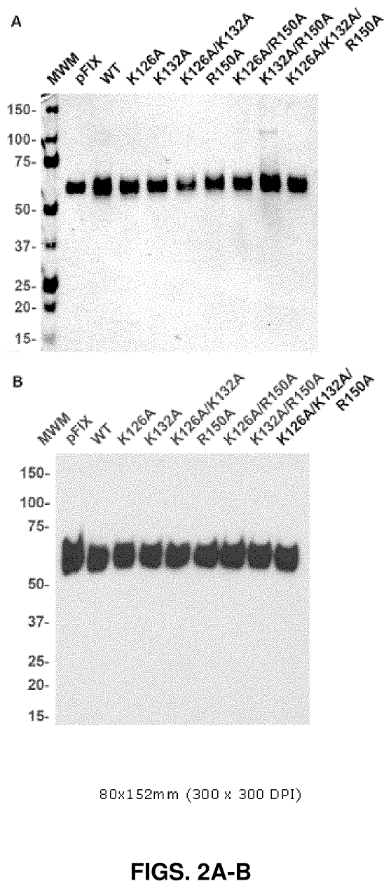

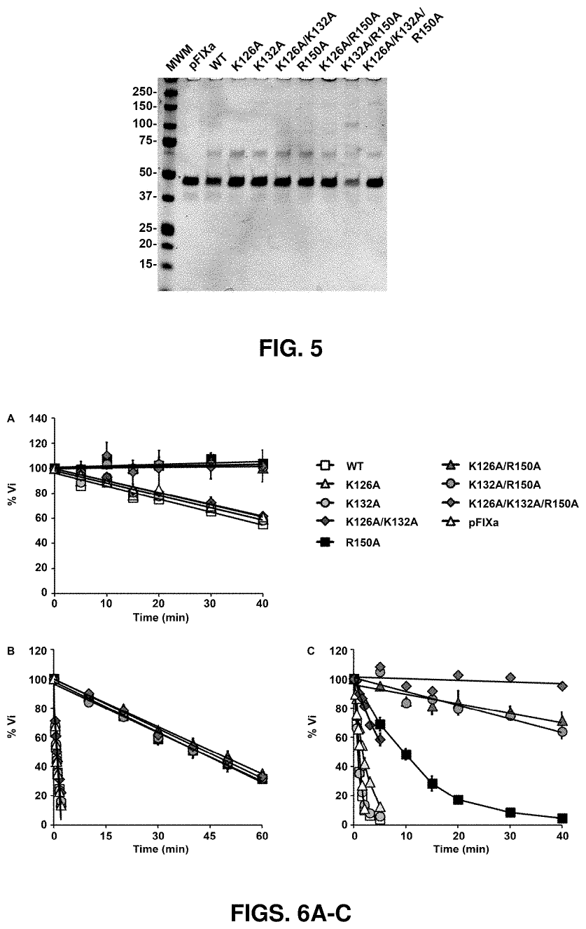

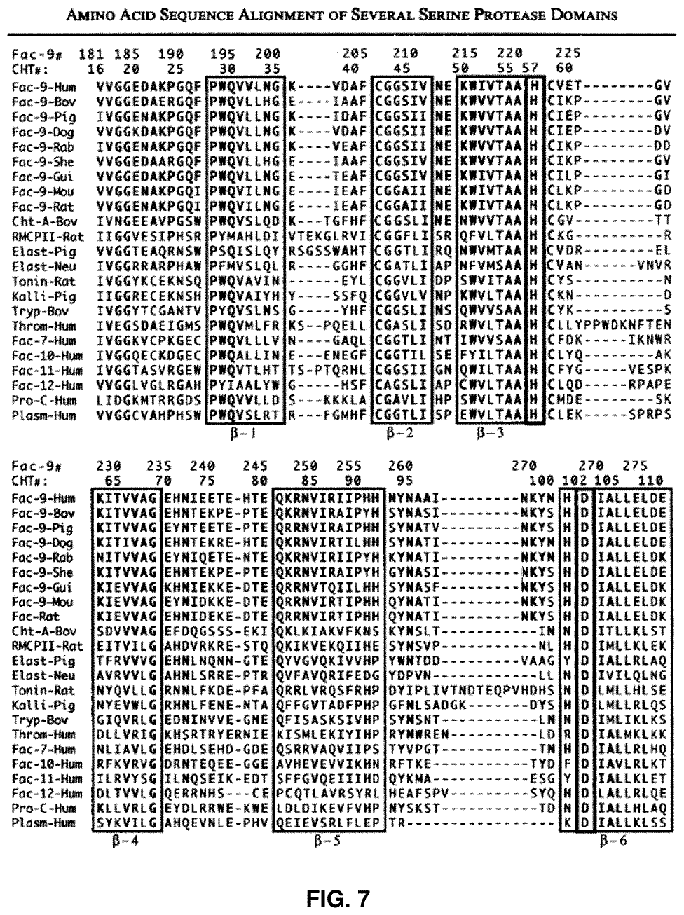

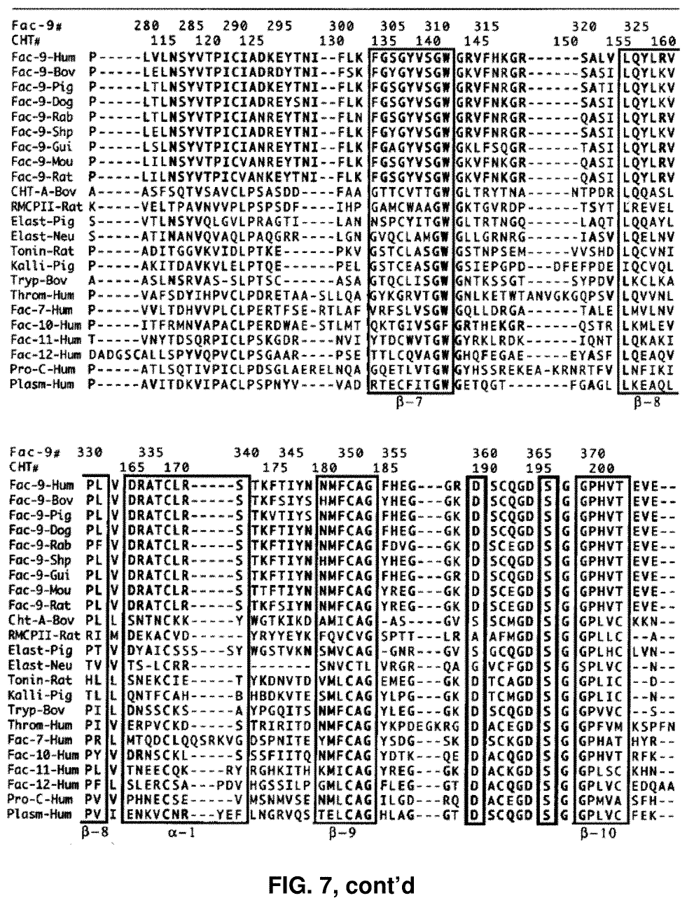

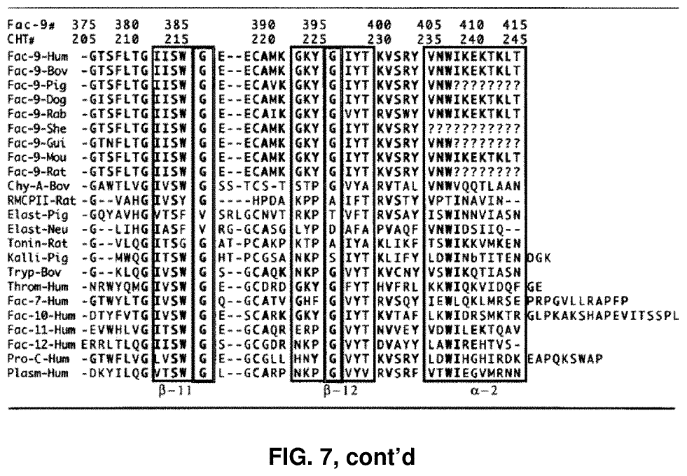

Alanine substitutions were introduced into the heparin- (K126 and K132) and antithrombin-binding (R150) exosites on the FIX protease domain (FIG. 1). rFIX proteins were expressed in HEK293 cells over-expressing VKOR to improve the yield of fully γ-carboxylated protein (Sun et al., 2005). rFIX proteins were purified to homogeneity from conditioned media (Yuan et al., 2005), exhibited high purity by 10% SDS-PAGE (non-reducing conditions) stained with Coomassie Blue (FIG. 2A), and a single 56 kDa band was visible by Western blot (FIG. 2B). rFIX was activated to rFIXa with FXIa and active-site titrated with antithrombin as previously described (Yuan et al., 2005). The protease forms also exhibited high purity by Coomassie Blue staining with band at 45 kDa (>95%) and a minor contaminating rFIX band at 56 kDa (FIG. 5).

Coagulant Activity of rFIX(a) Proteins.

Coagulant activity was determined for both zymogen and protease in FIX-deficient plasma and normalized to recombinant WT protein activity (100%) (Table 1). For the zymogens, coagulant activity of pFIX was similar to the WT protein, rFIX K126A and R150A demonstrated mild reduction (˜60% WT activity), and rFIX K132A demonstrated moderate reduction (˜30%). Dual heparin mutations in the heparin exosite (K126A/K132A) further reduced coagulant activity, and the triple mutant K126A/K132A/R150A demonstrated the lowest activity (<10%). Combined exo site mutations (K126A/R150A, K132A/R150A) demonstrated moderate (˜30%) and mild (˜75%) decreases in coagulant activity, respectively. For the proteases, the coagulant activity of pFIXa was similar to the WT protein, rFIXa K126A was moderately reduced (˜40%), and rFIXa K132A and R150A demonstrated minimal or mild reduction (˜91% or 77%), respectively. rFIX possessing the dual mutations in the heparin exosite (K126A/K132A, K126A/K132A/R150A) demonstrated markedly reduced coagulant activity relative to WT (˜10%). However, combined exosite mutations (K126A/R150A, K132A/R150A) resulted in moderate or mild reductions in coagulant activity (˜25% or 85%), respectively.

Effect of rFIX(a) Exosite Mutations on Plasma Thrombin Generation Activity.

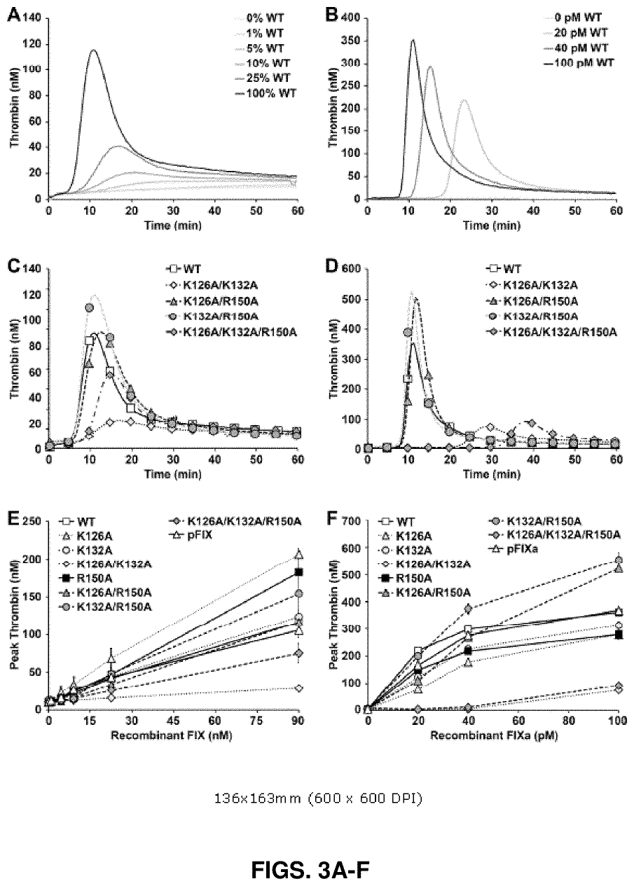

The ability of rFIX to support TF-triggered thrombin generation was examined in FIX-deficient plasma supplemented with 1-100% rFIX (FIG. 3A). All zymogens demonstrated a dose dependent increase in thrombin generation, expressed as peak thrombin concentration. When the mean peak thrombin concentration generated in the presence of 100% levels (90 nM) of FIX was compared (Table 2, FIGS. 3C and 3E), plasma-derived, WT and rFIX K132A were similar, while rFIX K126A and R150A demonstrated increased (1.6- to 1.8-fold) peak thrombin generation relative to WT. rFIX containing dual mutations in the heparin-binding exosite (K126A/K132A, K126A/K132A/R150A) exhibited significant reductions (0.2-0.6 fold) in peak thrombin generation. In contrast, rFIX containing combined exosite mutations (K126A/R150A, K132A/R150A) demonstrated similar or increased (1.0- to 1.3-fold) peak thrombin levels relative to WT (Table 2, FIG. 3C). Similarly, peak thrombin concentration increased in a relatively linear fashion with increasing FIX, with similar responses for pFIX and rFIX WT, K132A and K126A/R150A. The dose responses for rFIX K126A/K132A and K126A/K132A/R150A were shifted mildly and markedly to the right, respectively; while rFIX K132A/R150A, R150A and K126A shifted progressively to the left (FIG. 3E).

Similarly, all proteases, rFIXa (20-100 pM), demonstrated a dose-dependent increase in thrombin generation, with more pronounced shortening of the lag phase than observed for the zymogens (FIG. 3B). When peak thrombin concentrations triggered by 100 pM FIXa were compared, plasma-derived and WT rFIXa were similar, and single mutations in rFIX K126A, K132A and R150A demonstrated modest reductions in peak thrombin concentration (0.8 to 0.9-fold) relative to WT (Table 2). The dual mutations in the heparin-binding exosite (K126A/K132A, K126A/K132A/R150A) significantly reduced (0.2-0.3 fold) peak thrombin generation relative to WT. In contrast, the combined exosite mutations in rFIX K126A/R150A and K132A/R150A demonstrated significantly increased (1.4- to 1.5-fold) peak thrombin levels relative to rFIX WT (Table 2, FIG. 3D). Consistent with these results, peak thrombin concentration increased with FIXa concentration in a similar fashion for pFIXa and rFIXa WT, K126A, K132A and R150A (FIG. 3F). In contrast, the dose response for rFIXa K126A/K132A and K126A/K132A/R150A were shifted markedly to the right, while the rFIXa K126A/R150A and K132A/R150A were shifted modestly to the left.

Effect of rFIXa Exosite Mutations on the Rate of Inhibition by Antithrombin.

The rate of FIXa inhibition by antithrombin was determined under pseudo-first order conditions with sampling to detect residual protease activity over time. In the absence of heparin, estimated rate constants for inhibition of pFIXa and the rFIXa WT, K126A, K132A and K126A/K132A were similar. In contrast, all rFIXa proteases containing the R150A mutation (R150A, K126A/R150A, K132A/R150A, K126A/K132A/R150A) demonstrated negligible inhibition (estimated rate constants <0.3×10³ M⁻¹ min⁻¹) over the time course of the experiment (Table 3, FIG. 6A).

The rate of FIXa inhibition by antithrombin was also determined in the presence of a therapeutically relevant concentration of synthetic pentasaccharide Fondaparinux (460 nM) over 2-5 min for most of the proteases (Bauer et al., 2002; Paolucci et al., 2002). Fondaparinux accelerated the rate of antithrombin inhibition for pFIXa and rFIXa WT by approximately 64- and 78-fold, respectively (Table 3, FIG. 6B). rFIXa WT, K126A, K132A and K126A/K132A demonstrated similar rates of inhibition by antithrombin, demonstrating that the heparin-binding exosite does not significantly contribute to interaction with the antithrombin-pentasaccharide complex. In contrast, rFIXa containing the R150A mutation (R150A, K126A/R150A, K132A/R150A, K126A/K132A/R150A) demonstrated ˜49-fold reduction in the rate of antithrombin inhibition relative to WT.

A sub-saturating concentration of UFH (˜0.24 U/mL), selected to allow accurate rate determination in the discontinuous assay, resulted in an approximately 80-fold increase in estimated rate constants for antithrombin inhibition of pFIXa and rFIXa WT. Rate constants for inhibition of rFIXa K132A, K126A and K126A/K132A were reduced 1.2-, 2.8-, and over 10-fold relative to WT, respectively, under these conditions (Table 3, FIG. 6C). rFIXa R150A also demonstrated a marked reduction in the rate of antithrombin-heparin inhibition, with an estimated inhibition rate constant over 16-fold less than the WT protease. Combining heparin exosites mutations with R150A resulted in additional resistance to inhibition. Estimated rate constants for inhibition of rFIXa K126A/R150A, K132A/R150A and K126A/K132A/R150A by antithrombin were reduced 200-fold, 100-fold and over 580-fold, respectively, relative to WT. Thus, combined mutations in the heparin- and antithrombin-binding exosites resulted in synergistic reductions in the rate of inhibition by antithrombin-heparin.

Effect of rFIXa Exosite Mutations on Protease Half-Life in FIX-Deficient Plasma.

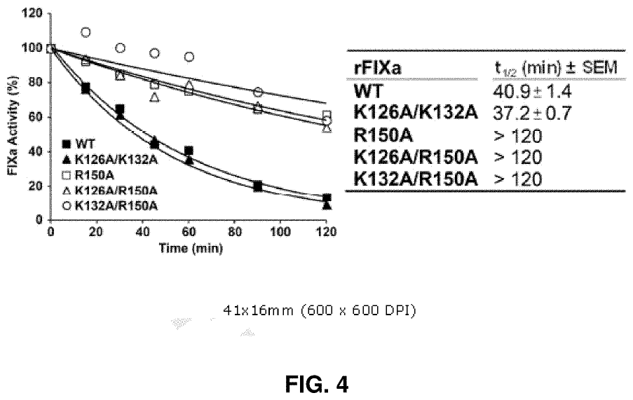

The effect of these mutations on the persistence of rFIXa activity in plasma was analyzed by spiking 50-400 pM rFIXa into immuno-depleted FIX-deficient plasma and determining residual rFIXa activity over time with the ETGA. rFIXa WT demonstrated a remarkably long half-life in FIX-deficient plasma (40.9±1.4 min). The double mutation in the heparin-binding exosite (K126A/K132A) had negligible effect on this result. In contrast, recombinant proteases containing the R150A mutation (rFIXa R150A, K126A/R150A, K132A/R150A) demonstrated markedly prolonged half-lives (>2 hr) (FIG. 4).

TABLE 1
Coagulant Activity of rFIX and rFIXa proteins

| | FIX | FIXa |
|---|---|---|
| Mutation | (% ± SEM) | (% ± SEM) |
| WT | 100.0 ± 7.1 | 100.0 ± 6.1 |
| K126A | 63.3 ± 2.3 | 39.5 ± 2.4 |
| K132A | 30.9 ± 1.0 | 91.4 ± 1.6 |
| K126A/K132A | 20.6 ± 9.2 | 9.3 ± 0.6 |
| R150A | 62.4 ± 4.0 | 77.1 ± 5.8 |
| K126A/R150A | 27.0 ± 2.0 | 25.3 ± 2.8 |
| K132A/R150A | 75.8 ± 3.4 | 84.9 ± 2.7 |
| K126A/K132A/R150A | 7.3 ± 3.8 | 10.8 ± 0.6 |
| pFIXa | 105.1 ± 2.8 | 98.4 ± 11.4 |

APTT-based clotting activity expressed as proportion of rFIX(a) WT protein (%). Coagulant activity of 90 nM rFIX and 250 pM rFIXa constructs were determined by APTT assay in FIX-deficient plasma. pFIX and pFIXa activities were similarly determined for comparison. (mean±SEM, n=3-5).

TABLE 2
Plasma thrombin generation by rFIX(a) in FIX-deficient plasma

| | rFIX (90 nM) | | rFIXa (100 pM) | |
|---|---|---|---|---|
| Construct | nM ± SEM | % ± SEM | pM ± SEM | % ± SEM |
| WT | 116.8 ± 3.7 | 100.0 ± 2.3 | 361.4 ± 18.6 | 100.0 ± 4.5 |
| K126A | 207.1 ± 4.7 | 177.24 ± 4.1 | 279.1 ± 16.2 | 77.2 ± 4.5 |
| K132A | 123.9 ± 5.7 | 106.0 ± 4.9 | 313.5 ± 12.3 | 86.8 ± 3.4 |
| K126A/ | 28.8 ± 2.1 | 24.6 ± 1.8 | 75.1 ± 3.3 | 20.8 ± 0.9 |
| K132A | | | | |
| K150A | 182.4 ± 32.4 | 156.1 ± 27.7 | 279.9 ± 19.0 | 77.5 ± 5.3 |
| K126A/ | 116.9 ± 16.2 | 100.0 ± 13.9 | 522.7 ± 12.2 | 144.6 ± 3.4 |
| R150A | | | | |
| K132A/ | 154.3 ± 27.7 | 132.1 ± 23.7 | 554.2 ± 26.9 | 153.4 ± 7.3 |
| R150A | | | | |
| K126A/ | 75.2 ± 12.6 | 64.4 ± 10.8 | 92.7 ± 5.4 | 25.7 ± 1.5 |
| K132A/ | | | | |
| R150A | | | | |
| pFIXa | 105.5 ± 10.0 | 90.3 ± 8.6 | 368.9 ± 16.1 | 102.1 ± 4.5 |

Peak thrombin concentration (nM) and % WT activity (mean±SEM, n=3-4) were determined in: a) the TF-triggered TGA assay the presence of 90 nM rFIX or b) FIXa-initiated TGA in the presence of 100 pM rFIXa. pFIX and pFIXa were similarly tested for comparison.

TABLE 3
Estimated rate constants (k2) for inhibition of
FIXa by ATIII in the absence or presence heparins

| | ATIII | ATIII/Fond | | ATIII/UFH | |
|---|---|---|---|---|---|
| | k₂ (10³ | k₂ (10³ | | k₂ (10³ | |
| | M⁻¹ | M⁻¹ | | M⁻¹ | |
| Protease | min⁻¹) ± | min⁻¹) ± | Fold- | min⁻¹) ± | Fold- |
| (FIXa) | SEM | SEM | Increase | SEM | Increase |
| WT | 3.1 ± 0.2 | 197.4 ± 2.1 | 63.7 | 260.0 ± 9.0 | 83.9 |
| K126A | 2.8 ± 0.1 | 197.9 ± 8.9 | 70.7 | 93.4 ± 2.3 | 33.4 |
| K132A | 2.8 ± 0.0 | 195.0 ± 4.5 | 69.6 | 224.4 ± 6.7 | 80.1 |
| K126A/ | 2.6 ± 0.1 | 158.5 ± 3.7 | 61.0 | 25.4 ± 2.2 | 9.8 |
| K132A | | | | | |
| R150A | <0.3 | 4.3 ± 0.4 | 14.3 | 15.7 ± 2.9 | 52.3 |
| K126A/ | <0.3 | 3.8 ± 0.1 | 12.7 | 1.3 ± 0.5 | 4.3 |
| R150A | | | | | |
| K132A/ | <0.3 | 4.1 ± 0.3 | 13.7 | 2.5 ± 0.2 | 8.3 |
| R150A | | | | | |
| K126A/ | <0.3 | 4.1 ± 0.5 | 13.7 | 0.4 ± 0.2 | 1.3 |
| K132A/ | | | | | |
| R150A | | | | | |
| pFIXa | 2.7 ± 0.1 | 211.3 ± 3.4 | 78.3 | 205.8 ± 16.6 | 76.2 |

rFIXa (292.5 nM) and ATIII (4.5 μM) were incubated in the presence of fondaparinux (460 nM) or unfractionated heparin (0.24 U/mL) with sampling over time into a reaction containing final concentrations of 250 μM Pefafluor® FIXa, 30% ethylene glycol, and 1 mg/ml Polybrene® in tenase buffer to determine residual protease activity. The estimated rate constant determined under pseudo-first order conditions was expressed as the mean value (n=3-4, ±SEM) and as a fold-increase over the baseline rate constant for each protease. There was no significant loss of FIXa activity in the absence of antithrombin over the duration of assays (not shown). Representative inhibition curves for select rFIXa constructs are shown in FIGS. 6A-C.

TABLE 4
Primers used for generating the rFIX(a) mutations

| Mutation | Primer |
|---|---|
| K126A | 5′-CCT ATT TGC ATT GCT GAC GCG GAA TAC |
| forward: | ACG AAC ATC TTC C-3′ |
| K126A | 5′-G GAA GAT GTT CGT GTA TTC CGC GTC AGC |
| reverse: | AAT GCA AAT AGG-3′ |
| K132A | 5′-CG AAC ATC TTC CTC GCA TTT GGA TCT |
| forward: | GGC TAT GTA AGT GG-3′ |
| K132A | 5′-C ATA GCC AGA TCC AAA TGC GAG GAA GAT |
| reverse: | GTT CGT GTA TTC C-3′ |
| R150A | 5′-GA GTC TTC CAC AAA GGG GCA TCA GCT |
| forward: | TTA GTT CTT CAG-3′ |
| R150A | 5′-CTG AAG AAC TAA AGC TGA TGC CCC TTT |
| reverse: | GTG GAA GAC TC-3′ |

Codon for mutated amino acid is in bold.

TABLE 5
Detection of FIXa activity in zymogen preparations

| rFIXa Preparation | pM rFIXa |
|---|---|
| WT | 7.2 |
| K126A | 5.7 |
| K132A | 17.9 |
| K126A/K132A | 0.9 |
| R150A | 38.3 |
| K126A/R150A | 1.0 |
| K132A/R150A | 3.4 |
| K126A/K132A/R150A | 4.0 |
| pFIX | 6.4 |

The presence of FIXa in zymogen preparations was assessed with the ETGA after supplementation of FIX-deficient plasma with 90 nM FIX (100%). All zymogen preparations demonstrated <10 pM protease except for FIX K132A (˜18 pM) and R150A (˜38 pM) (n=1-2).

TABLE 6
Pefafluor FIXa ® cleavage by FIXa

| | Activity | |
|---|---|---|
| rFIXa | (% ± SEM) | t-Test |
| WT | 100.0 ± 1.8 | 1.00 |
| K126A | 96.9 ± 7.5 | 0.71 |
| K132A | 103.6 ± 1.3 | 0.22 |
| K126A/K132A | 97.7 ± 5.0 | 0.70 |
| R150A | 121.3 ± 7.6 | 0.04 |
| K126A/R150A | 99.2 ± 0.9 | 0.90 |
| K132A/R150A | 109.9 ± 1.7 | 0.01 |
| K126A/K132A/R150A | 72.8 ± 3.1 | 0.00 |
| BeneFIXa | 104.5 ± 4.6 | 0.42 |
| pFIXa | 109.4 ± 3.8 | 0.07 |

The ability rFIXa and pFIXa (0-80 nM) to cleave Pefafluor FIXa® (CH3SO2-D-CHG-Gly-Arg-AMC.AcOH) was assessed in a reaction containing final concentrations of 30% ethylene glycol, 250 μM Pefafluor IXa® and 1 mg/mL Polybrene® in tenase buffer at RT. Initial rates of substrate hydrolysis were determined by the fluorescence change (360/40-nm-excitation, 460/40-nm-emission) over 10 min. Vi was calculated by plotting fluorescent intensity versus time and determining the initial slope. Vi was plotted versus rFIXa concentration, and slope of the standard curve determined for each protein. Relative slope values for the recombinant FIXa proteins were expressed as percent activity relative to WT (n=3-5, ±SEM).

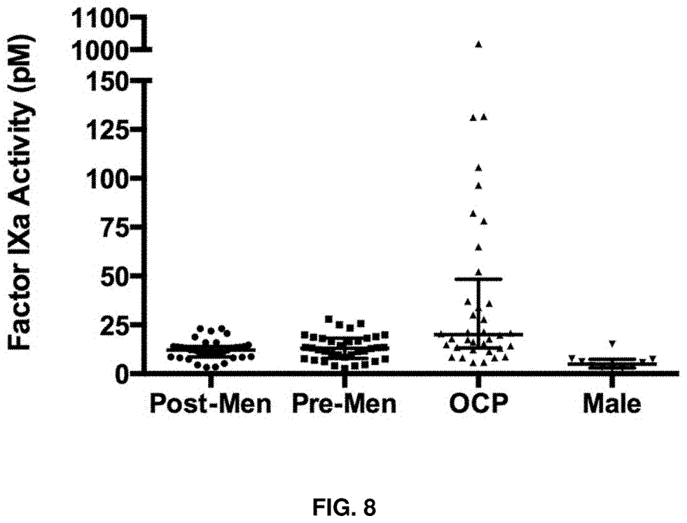

TABLE 7
Descriptive statistics for factor IXa activity determinations
in volunteer blood donors (data presented in FIG. 8)

| | | Pre- | Pre- | |
|---|---|---|---|---|
| | Post- | menopausal | menopausal | |
| | menopausal | Females (No | Females + | |
| | Females | OCP) | OCP | Males |
| Total number | 36 | 36 | 36 | 10 |
| Mean Age | 64.6 | 31.6 | 31.2 | 29.3 |
| Minimum | 3.35 | 2.74 | 5.73 | 2.60 |
| 25% Percentile | 8.62 | 7.86 | 13.19 | 3.01 |
| Median | 12.13 | 13.02 | 20.09 | 4.90 |
| 75% Percentile | 13.99 | 18.20 | 48.39 | 7.43 |
| Maximum | 23.14 | 27.90 | 1018.0 | 15.13 |
| Mean | 12.22 | 13.35 | 62.14 | 5.83 |
| SD | 4.99 | 6.48 | 167.5 | 3.76 |
| SEM | 0.83 | 1.08 | 27.92 | 1.19 |

### Example 3—Discussion

Selective mutagenesis of the regulatory exosites for heparin and antithrombin on human FIX(a) was performed, and the effect on traditional coagulant activity, the ability to support plasma thrombin generation, inhibition by antithrombin and protease plasma half-life was characterized. The results demonstrate that rFIX(a) proteins possessing combined exosite mutations unexpectedly preserved or enhanced plasma thrombin generation and synergistically reduced the rate of inhibition by antithrombin-heparin. The plasma half-life for FIXa activity was determined using a novel method capable of detecting physiologically relevant protease concentrations. The baseline plasma half-life of rFIXa was remarkably lengthy and further prolonged by the R150A mutation in the antithrombin-binding exosite. The phenotype of these rFIX(a) proteins (intact pro-coagulant function with defective regulation by antithrombin-heparan sulfate) should enhance the efficacy of hemophilia B therapy.

Comparison of APTT-based coagulant activity and plasma thrombin generation yielded significant differences for several of the mutant rFIX(a) proteins. The clotting endpoint in the APTT-based assay for the zymogen is: 1) dependent on FXIa activation of rFIX, 2) relatively insensitive (compared to plasma thrombin generation) to rFIXa contamination and 3) occurs prior to peak thrombin generation in the presence of excess rFIXa and limiting FVIIIa (Sheehan and Lan, 1998). In contrast, plasma thrombin generation by the zymogen is: 1) dependent on rFIX activation by both TF-factor VIIa and FXIa, 2) sensitive to picomolar rFIXa contamination and 3) occurs in the presence of limiting rFIXa and excess FVIIIa (Lawson et al., 1994). Analysis of rFIXa is more direct than the zymogen, as it excludes the influence of rFIX activation and protease contamination. rFIXa K132A and R150A demonstrated mild proportionate reductions in coagulant activity and plasma thrombin generation relative to WT, suggesting largely intact pro-coagulant function. A disproportionate decrease in coagulant activity relative to thrombin generation was observed for rFIXa K126A, suggesting that the mildly reduced cofactor affinity for this protein (Misenheimer et al., 2007) is compensated by relative FVIIIa excess in the TGA. rFIXa K126A/K132A had markedly reduced coagulant activity and thrombin generation, likely the combined effect of these mutations on the overlapping protease-cofactor binding site (Misenheimer et al., 2007; Yuan et al., 2005; Miseheimer and Sheehan, 2010). In contrast, combination of single mutations in the heparin exosite with R150A variably reduced coagulant activity, but unexpectedly increased the magnitude of plasma thrombin generation for rFIXa K126A/R150A and K132A/R150A. Similarly, the triple mutant (K126A/K132A/R150A) showed a disproportionate increase in thrombin generation relative to coagulant activity. These unexpected results suggest that combined exosite mutations have synergistic effects on FX activation under the conditions present in the plasma TGA.

While TF-triggered plasma thrombin generation likely has more physiologic relevance than APTT-based coagulant activity, it is also more sensitive to rFIXa contamination of the zymogen, as a 1000-fold difference exists between active concentrations of zymogen and protease in this assay. rFIXa contamination of the zymogen preparations was assessed with the ETGA in FIX-deficient plasma. At 90 nM (100%) rFIX, all zymogens demonstrated <10 pM protease except for rFIX K132A (˜18 pM) and R150A (˜38 pM) (Table 5). Thus, thrombin generation activity (Table 2) is likely mildly or moderately over-estimated, respectively, for these zymogens. The higher thrombin generation activity demonstrated by rFIX K126A, K126A/R150A and K132A/R150A cannot be explained by protease contamination. Likewise, no significant protease contamination of the triple mutant (K126A/K132A/R150A) is present to explain the significantly enhanced thrombin generation activity relative to coagulant activity.

Single mutations in the antithrombin and heparin binding exosites of rFIXa had the expected effects on inhibition by antithrombin-heparin. Heparin-binding exosite mutations did not affect baseline or pentasaccharide-accelerated inhibition of rFIXa (Table 3), as this exosite does not contribute to the protease-antithrombin interaction. The dual heparin exosite mutations resulted in very modest slowing of inhibition, consistent with the conformational linkage of this exosite to the protease active site (Miseheimer et al., 2007). All proteases containing the R150A mutation demonstrated substantial reduction in baseline and pentasaccharide-accelerated inhibition, consistent with the critical role of this residue in the exosite-mediated interaction with antithrombin (Yang et al., 2003; Johnson et al., 2010). In the presence of UFH, rFIXa K132A and K126A exhibited mild to moderate reductions in the rate of inhibition by antithrombin relative to WT, consistent with their effects on direct inhibition of the intrinsic tenase complex by LMWH (Misenheimer and Sheehan, 2010), while combining these mutations in the heparin exosite demonstrated a synergistic reduction in the antithrombin inhibition rate. Consistent with pentasaccharide results, the R150A mutation substantially reduced the heparin-stimulated inhibition rate for antithrombin inhibition (Table 3). Notably, combining R150A with either mutation in the heparin-binding exosite resulted in a synergistic reduction in the inhibition rate. The inhibition rate for the triple mutant in the presence of heparin was substantially slower than the baseline rate for rFIXa WT in the absence of heparin. Thus, combined mutations in the heparin and antithrombin binding sites resulted in rFIXa that was highly resistant to inhibition by antithrombin-heparin. Similarly, the half-life of rFIXa WT in human plasma was compatible with the baseline rate of rFIXa inhibition in vitro and dramatically longer (˜41 min) than reported plasma half-lives for FXa and thrombin (˜1 min) (FIG. 4) (Ruhl et al., 2012; Marlu and Polack, 2012; Bunce et al., 2011). The plasma half-lives of the latter proteases reflect not only their relative rates of inhibition by antithrombin, but also the availability of alternative plasma inhibitors for FXa and thrombin (Narita et al., 1998; Fuchs and Pizzo, 1983; Jesty, J., 1986). While mutations in the heparin binding exosite had no effect in the absence of heparin, the R150A mutation markedly prolonged protease half-life, consistent with the role of antithrombin as the primary plasma inhibitor of FIXa (FIG. 4).

Infusion of rFIX into hemophilia B mice promotes hemostasis up to seven days (with plasma levels <1%) in a saphenous vein bleeding model, and the magnitude of this effect depends on relative affinity of rFIX for collagen IV, suggesting that this extravascular pool makes an important contribution to in vivo hemostasis (Gui et al., 2009; Feng et al., 2013; Gui et al., 2002]. Similar to anticoagulant heparan sulphate, collagen IV localizes predominantly to the basement membrane, suggesting that antithrombin and FIX may co-localize in the vessel wall (Cheung et al., 1996; de Agostini et al., 1990; Shekhonin et al., 1985). Additional FIX binding sites exist, as ˜80% of injected protein is sequestered in the liver in a largely collagen IV independent manner (Gui et al., 2002). FIX(a) demonstrates increased affinity for heparin/heparan sulfate relative to other coagulation factors (Bajaj et al., 1981), and liver heparan sulfate has been implicated in the clearance of lipoproteins directly and proteases/protease-inhibitor complexes by lipoprotein receptor-related protein (LRP) family members (Standford et al., 2009; Bishop et al., 2008; Ho et al., 1997; Kounnas et al., 1995; 1996). Further, surface proteoglycans and LRP contribute to the intracellular degradation and clearance of FIXa, involving a binding site that overlaps with the heparin exosite (Rohlena et al., 2003; Neels et al., 2000). Given the lack of alternative plasma inhibitors, co-localization with antithrombin in the vascular wall and contribution of the heparin exosite to cellular clearance by LRP, rFIX with combined mutations in the antithrombin and heparin exosites should demonstrate markedly prolonged in vivo activity.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods, and in the steps or in the sequence of steps of the methods described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VIII. REFERENCES

The following references, to the extent that they provide exemplary procedural or other details supplementary to those set forth herein, are specifically incorporated herein by reference.
U.S. Pat. No. 5,440,013
U.S. Pat. No. 5,446,128
U.S. Pat. No. 5,475,085
U.S. Pat. No. 5,597,457
U.S. Pat. No. 5,604,251
U.S. Pat. No. 5,618,914
U.S. Pat. No. 5,670,155
U.S. Pat. No. 5,672,681
U.S. Pat. No. 5,674,976
U.S. Pat. No. 5,710,245
U.S. Pat. No. 5,790,421
U.S. Pat. No. 5,840,833
U.S. Pat. No. 5,859,184
U.S. Pat. No. 5,889,155
U.S. Pat. No. 5,929,237
U.S. Pat. No. 6,093,573
U.S. Pat. No. 6,261,569
U.S. Pat. No. 7,183,059
U.S. Pat. No. 7,192,713
U.S. Publication Application No. 2005/0015232
Bajaj and Birktoft, Methods Enzymol. 1993, 22:96-128).
Capaldi et al., Biochem. Biophys. Res. Comm., 1977, 74(2):425-433.
Klaassen, In: The Pharmacological Basis of Therapeutics, Goodman and Gilman, Eds., Pergamon Press, 8^{th} Ed., 1990.
Peptide Synthesis, 1985
Physicians Desk Reference
Protective Groups in Organic Chemistry, 1973
Protein NMR Spectroscopy, Principles and Practice, 1996
Remington's Pharmaceutical Sciences, 15^{th} ed., 1035-1038 and 1570-1580, Mack Publishing Company, P A, 1980.
Solid Phase Peptide Synthelia, 1984
The Merck Index, 11th Edition.
Manco-Johnson, M J., Seminars in Hematology, 2003; 40: 3-9.
Manco-Johnson et al., N Engl J Med., 2007; 357: 535-44.
Ostergaard et al., Blood, 2011; 118: 2333-41.
Martinowitz et al., Thromb Res., 2013; 131 Suppl 2: S11-4.
Powell et al., N Engl J Med., 2013; 369: 2313-23.
Bjorkman et al., European J Clinical Pharmacology, 1994; 46: 325-32.
Stern et al., Br J Haematol., 1987; 66: 227-32.
Stern et al., Proc Natl Acad Sci USA., 1983; 80: 4119-23.
Cheung et al., Proc Natl Acad Sci USA., 1996; 93: 11068-73.
Gui et al., J Thromb Haemost., 2009; 7: 1843-51.
Feng et al., J Thromb Haemost., 2013, 11(12):2176-8.
Rand et al., Blood, 1996; 88: 3432-45.
Brandstetter et al., Proc Natl Acad Sci USA, 1995; 92: 9796-800.
Hopfner et al., Structure Fold Des., 1999; 7: 989-96.
Duffy et al., Journal of Biological Chemistry, 1992; 267: 17006-11.
Zogg et al., Structure, 2009; 17: 1669-78.
Fay et al., Journal of Biological Chemistry, 1996; 271: 6027-32.
Fuchs et al., Journal of Clinical Investigation, 1984; 73: 1696-703.
de Agostini et al., J Cell Biol., 1990; 111: 1293-304.
Krishnaswamy, S., J. Thromb. Haemost., 2005; 3: 54-67.
Misenheimer et al., Biochemistry, 2007; 46: 7886-95.
Yuan et al., Biochemistry, 2005; 44: 3615-25.
Yang et al., J Biol Chem., 2002; 277: 50756-60.
Yang et al., J Biol Chem., 2003; 278: 25032-8.
Sheehan et al., Biochemistry, 2003; 42: 11316-25.
Bedsted et al., Biochemistry, 2003; 42: 8143-52.
Misenheimer and Sheehan, Biochemistry. 2010; 49: 9997-10005.
Johnson et al., Proc Natl Acad Sci USA, 2010; 107: 645-50.
MacDonald et al., Biochimica et Biophysica Acta, 1991; 1061: 297-303.
Buyue et al., Blood, 2008; 112: 3234-41.
Sun et al., Blood, 2005; 106: 3811-5.
Bauer et al., Cardiovasc Drug Rev., 2002; 20: 37-52.
Paolucci et al., Clin Pharmacokinet., 2002; 41 Suppl 2: 11-8.
Sheehan and Lan et al., Blood, 1998; 92: 1617-25.
Lawson et al., J Biol Chem., 1994; 269: 23357-66.
Ruhl et al., Thromb Haemost., 2012; 107: 848-53.
Marlu and Polack, Haematologica, 2012; 97: 1165-72.
Bunce et al., Blood, 2011; 117: 290-8.
Narita et al., Blood, 1998; 91: 555-60.
Fuchs and Pizzo, J Clin Invest., 1983; 72: 2041-9.
Jesty, J., J Biol Chem., 1986; 261: 10313-8.
Gui et al., Blood, 2002; 100: 153-8.
Shekhonin et al., Collagen and Related Research, 1985; 5:355-68.
Bajaj et al., Preparative Biochemistry, 1981; 11: 397-412.
Stanford et al., J Clin Invest., 2009; 119: 3236-45.
Bishop et al., Current Opinion Lipidology, 2008; 19: 307-13.
Ho et al., J Biol Chem., 1997; 272: 16838-44.
Kounnas et al., J Biol Chem., 1996; 271: 6523-9.
Kounnas et al., J Biol Chem., 1995; 270: 9307-12.
Rohlena et al., J Biol Chem., 2003; 278: 9394-401.
Neels et al., Blood 2000; 96: 3459-65.

1-10. (canceled)

11. A method of treating hemophilia or hemorrhagic disease comprising administering, to a subject in need thereof, a Factor IX protein comprising a R→A substitution at residue 150 of the native sequence and either or both (a) a K→A substitution at residue 126 of the native sequence or (b) a K→A substitution at residues 132 of the native sequence, as defined by the chymotrypsinogen numbering system for the protease domain.

12. The method of claim 11, wherein said protein is full length uncleaved Factor IX.

13. The method of claim 11, wherein said protein is lacks the signal sequence of full length Factor IX.

14. The method of claim 11, wherein said protein is cleaved to Factor IXa.

15. The method of claim 11, wherein the sequence of said protein comprises SEQ ID NO: 2.

16. The method of claim 15, wherein the sequence of said protein comprises SEQ ID NO: 4.

17. The method of claim 16, wherein the sequence of said protein comprises SEQ ID NO: 6.

18. The method of claim 11, wherein the sequence of said protein consists of SEQ ID NO: 2.

19. The method of claim 18, wherein the sequence of said protein consists of SEQ ID NO: 4.

20. The method of claim 19, wherein the sequence of said protein consists of SEQ ID NO: 6.

21. The method of claim 11, wherein administering comprises intravenous delivery, subcutaneous delivery, or transdermal delivery.

22. The method of claim 21, wherein subcutaneous delivery comprises delivery through a pump or implantable depot device.

23. The method of claim 21, wherein said protein is formulated with one or more of L-histidine, sucrose, glycine, and/or a polysorbate.

24. The method of claim 11, wherein the hemophilia is hemophilia B.

25. The method of claim 11, wherein the hemophilia is congenital or acquired.

26-44. (canceled)
